(19) United States
(12) Patent Application Publication
(10) Pub. No.: US 2018/0299379 A1
(43) Pub. Date: Oct. 18, 2018

(54) HIGH-THROUGHPUT ABSORBANCE MEASUREMENTS OF SAMPLES IN MICROCAPILLARY ARRAYS

(71) Applicant: xCella Biosciences, Inc., Menlo Park, CA (US)

(72) Inventor: Bob CHEN, Menlo Park, CA (US)

(21) Appl. No.: 15/948,865

(22) Filed: Apr. 9, 2018

Related U.S. Application Data
(60) Provisional application No. 62/483,871, filed on Apr. 10, 2017.
(60) Provisional application No. 62/534,614, filed on Jul. 19, 2017.

(51) Int. Cl. G01N 21/59; C12Q 1/25; C12Q 1/02

(52) U.S. Cl. CPC G01N 21/59; C12Q 1/02; C12Q 1/25

(57) ABSTRACT

The present invention provides a method for measuring the amount of absorbance of a sample in a microcapillary based on measuring the absorbance in the sample.

### CROSS REFERENCE TO RELATED APPLICATIONS

This application claims the benefit under 35 U.S.C. § 119(e) of U.S. Provisional Application No. 62/483,871, filed on Apr. 10, 2017 and Provisional Application No. 62/534,614 filed on Jul. 19, 2017 which are hereby incorporated by reference in their entireties.

### BACKGROUND OF THE INVENTION

The analysis of biological samples, including the identification, characterization, and re-engineering of proteins, nucleic acids, carbohydrates, and other important biomolecules, has benefited greatly from the scaling up of sample numbers and the scaling down of sample sizes. For example, the two-dimensional microarrays of biological materials, such as DNA microarrays, have enabled the development of high-throughput screening methods involving multiplexed approaches for processing samples and detecting results.

While such techniques provide analytical information about a particular sample, for example the presence and potentially the amount of a particular biomolecule in a solution or the sequence of a particular nucleic acid or polypeptide, they typically do not allow for the recovery of a biological sample identified by the assay without inactivating or otherwise damaging the sample of interest. Moreover, methods that allow for retrieval are often based on the use of fluorescent or other tags.

Fluorescence and other methods that have been employed in the context of microarray assay technologies have their limitations. Cells and/or molecules must fluoresce so that they are capable of detection using such fluorescence methods. As such, these methods require labeling, adding extra time and effort for assay set-up and development. In the context of high throughput technologies, such extra time and effort can be significant, in particular when working with hundreds of thousands or even millions of samples.

With the present invention and disclosure, the inventors have developed a novel method for employing High-throughput absorbance measurements of samples in microcavity arrays, including microcapillary and microwell arrays. The present invention meets this need and provides a method for measuring the amount of absorbance of a sample in a microcapillary.

In this method, different samples are contained within a microcapillary array, where each microcapillary contains one sample. An overhead light source transmits light of a specific wavelength through the samples held in the microcapillary array. The samples in the array will absorb differing amounts of the light (depending on the concentration of the sample). The remaining light will pass through the array into the microscope objective to the detector. The differing amounts of transmitted light can be used to discriminate and characterize samples.

There is therefore a continuing need to develop improved microscale screening and analysis methods and systems with high throughput capabilities, and particularly methods and systems that enable analysis and recovery of samples without the need to pre-tag or pre-label the samples being analyzed. The methods described herein meet this need and can find use in many applications, including enzyme engineering, ELISA assays, stability assays, and cell growth measurements.

### BRIEF SUMMARY OF THE INVENTION

The present invention provides high-throughput methods for determining absorbance for multiple samples in a microcavity array, the method comprising:
i) transmitting light of a definable wavelength through samples contained in said microcavity array, wherein one sample is loaded into each microcavity within the array;
ii) measuring the light transmitted through said samples with a detector, wherein the light transmitted is measured for each individual sample within the array in order to obtain a light transmittance intensity for each individual sample within the array;
iii) comparing the light transmittance intensity obtained for each individual sample in step ii) to the light transmittance intensity for a control sample; and
iv) calculating the absorbance of each individual sample in the array based on the comparison in step iii) in order to determine spectrometric differences between said samples.

In some embodiments, the light transmittance intensity is measured by the following formula:

$$T = \frac{{Intensity}_{sample}}{{Intensity}_{control}}$$

In some embodiments, the light transmittance intensity is measured by the following formula:

$$T = \frac{{Intensity}_{sample}}{{Intensity}_{blank}} .$$

In some embodiments, the light transmittance intensity is measured by the following formula:

$$T \% = \frac{{Intensity}_{sample}}{{Intensity}_{control}} * 100$$

In some embodiments, the light transmittance intensity is measured by the following formula:

$$T \% = \frac{{Intensity}_{sample}}{{Intensity}_{blank}} * 100.$$

In some embodiments, the light transmittance intensity is measured by the following formula:

$$T = \frac{{Intensity}_{sample}}{{Intensity}_{average}} .$$

In some embodiments, the light transmittance intensity is measured by the following formula:

$$T \% = \frac{{Intensity}_{sample}}{{Intensity}_{average}} * 100.$$

In some embodiments, the absorbance is calculated by the following formula:

A=−log₁₀T.

In some embodiments, the absorbance is calculated by the following formula:

A=2−log₁₀3T%.

In some embodiments, the method further comprises using said absorbance to determine one or more spectrometric characteristics.

In some embodiments, the spectrometric characteristics are selected from the group consisting of concentration, enzyme activity, enzyme-substrate interaction, receptor-ligand binding, affinity binding, stability, and cell growth.

In some embodiments, the enzyme activity results are based on a colorimetric assay.

In some embodiments, the concentration or cell growth results are based on a densitometric assay.

In some embodiments, the colorimetric assay is an enzyme based light absorbing assay.

In some embodiments, the densitometric assay is an assay wherein light is blocked by one or more materials in the sample.

In some embodiments, the material comprises one or more proteins, polypeptides, nucleic acid, small molecules, dyes, and/or cells.

In some embodiments, the method further comprises loading one sample into each microcavity prior to light transmission in step i).

In some embodiments, the microcavity is a microcapillary or a microwell.

In some embodiments, the light transmitted through said sample is detected by a microscope objective detector.

In some embodiments, the transmitted light is generated by a light source with a selectable wavelength.

In some embodiments, the transmitted light source is a high power plasma light source.

In some embodiments, the light source is a monochromatic light source. In some embodiments, the light source is coupled to a monochromator.

In some embodiments, the light source is coupled to one or more filters. In some embodiments, the light source is coupled to 1, 2, 3, 4, 5, or 6 filters.

In some embodiments, the sample comprises a biological material.

In some embodiments, the sample comprises proteins, polypeptides, nucleic acid, and/or cells. In some embodiments, the proteins or polypeptides are selected from the group consisting of enzymes, ligands, and receptors.

In some embodiments, the measurement in step ii) occurs simultaneously for all the samples.

In some embodiments, the detector is a camera. In some embodiments, the camera is a black and white camera. In some embodiments, the camera is a color camera.

In some embodiments, the detector is a photodiode.

In some embodiments, the detector is a photodiode, and the method further comprises imaging the location of each microcavity before or after step ii).

In some embodiments, the measurements in step ii) are performed in real time.

In some embodiments, the measurements in step ii) are performed on the same samples as part of a time course.

In some embodiments, the microarray comprises at least 100,000 samples.

In some embodiments, the sample volume is less than 500 nL.

In some embodiments, the method further comprises detecting more than spectrometric characteristics.

In some embodiments, the method further comprises detecting transmittance and fluorescence.

The present invention also provides a high-throughput microscope system for use in measuring the absorbance for multiple samples in a microcavity array, the microscope system comprising:
i) a light source unit comprising at least one light source capable of transmitting light of a definable wavelength through samples contained in said microcavity array, wherein one sample is loaded into each microcavity within the array;
ii) a detection unit comprising at least one detector capable of detecting the light transmitted through said samples, wherein the light transmitted is measured for each individual sample within the array in order to obtain a light transmittance intensity for each individual sample within the array;
iii) an optical train for directing the one or more illumination and/or excitation lights from the light source unit to the sample and for directing the transmitted light from the sample to the detection unit; and
iv) a control unit for controlling the light source unit and the detection unit; wherein, optionally the control unit is capable of:
   a) comparing the light transmittance intensity obtained for each individual sample in step ii) to the light transmittance intensity for a control sample; and
   b) calculating the absorbance of each individual sample in the array based on the comparison in step a) in order to determine differences between said samples.

In some embodiments of the high-throughput microscope system, the light transmittance intensity is measured by the following formula:

$$T = \frac{{Intensity}_{sample}}{{Intensity}_{control}} .$$

In some embodiments of the high-throughput microscope system, the light transmittance intensity is measured by the following formula:

$$T \% - \frac{{Intensity}_{sample}}{{Intensity}_{control}} * 100.$$

In some embodiments of the high-throughput microscope system, the light transmittance intensity is measured by the following formula:

$$T = \frac{{Intensity}_{sample}}{{Intensity}_{blank}} .$$

In some embodiments of the high-throughput microscope system, the light transmittance intensity is measured by the following formula:

$$T \% = \frac{{Intensity}_{sample}}{{Intensity}_{blank}} * 100.$$

In some embodiments of the high-throughput microscope system, the light transmittance intensity is measured by the following formula:

$$T = \frac{{Intensity}_{sample}}{{Intensity}_{average}} .$$

In some embodiments of the high-throughput microscope system, the light transmittance intensity is measured by the following formula:

$$T \% - \frac{{Intensity}_{sample}}{{Intensity}_{average}} * 100.$$

In some embodiments of the high-throughput microscope system, the absorbance is calculated by the following formula:

A=−log₁₀T.

In some embodiments of the high-throughput microscope system, the absorbance is calculated by the following formula:

A=2−log₁₀T%.

In some embodiments of the high-throughput microscope system, the light transmitted through said sample is detected by a microscope objective detector.

In some embodiments of the high-throughput microscope system, the transmitted light is generated by a light source with a selectable wavelength.

In some embodiments of the high-throughput microscope system, the transmitted light source is a high power plasma light source.

In some embodiments of the high-throughput microscope system, the light source is a monochromatic light source. In some embodiments of the high-throughput microscope system, the light source is coupled to a monochromator.

In some embodiments of the high-throughput microscope system, the light source is coupled to one or more filters. In some embodiments of the high-throughput microscope system, the light source is coupled to 1, 2, 3, 4, 5, or 6 filters.

In some embodiments of the high-throughput microscope system, the detector is a camera. In some embodiments of the high-throughput microscope system, the camera is a black and white camera. In some embodiments of the high-throughput microscope system, the camera is a color camera.

In some embodiments of the high-throughput microscope system, the detector is a photodiode.

### BRIEF DESCRIPTION OF THE DRAWINGS

FIG. 1 provides a schematic of High-throughput absorbance measurements of samples in microcapillary arrays.

FIG. 2 provides data showing Trypan Blue Solution with a max absorbance: 607 nm. Filter used: 620+/−30 nm. A 2-fold concentration dilution series was prepared and analyzed.

FIG. 3 provides a plot of the absorbance and concentration data shown in FIG. 2.

FIG. 4 provides a graph of absorbance spectrum for 4 absorbing solutions (dyes). These 4 dyes will be measured with 4 filter cubes: filter cube 1 (350/50 nm filter), filter cube 2 (475/40 nm filter), filter cube 3 (525/45 nm filter), and filter cube 4 (620/60 nm filter).

FIG. 5A-FIG. 5B provides a plot of the relationship between the 350 nm/50 nm filter and the absorbance spectrums of the 4 dyes, representative images of the 4 dyes in microcapillaries using the 350 nm/50 nm filter, and a graph of the quantified light intensity.

FIG. 6A-FIG. 6B provides a plot of the relationship between the 475 nm/40 nm filter and the absorbance spectrums of the 4 dyes, representative images of the 4 dyes in microcapillaries using the 475 nm/40 filter, and a graph of the quantified light intensity.

FIG. 7A-FIG. 7B provides a plot of the relationship between the 525 nm/45 nm filter and the absorbance spectrums of the 4 dyes, representative images of the 4 dyes in microcapillaries using the 525 nm/45 filter, and a graph of the quantified light intensity.

FIG. 8A-FIG. 8B provides a plot of the relationship between the 620 nm/60 nm filter and the absorbance spectrums of the 4 dyes, representative images of the 4 dyes in microcapillaries using the 620 nm/60 filter, and a graph of the quantified light intensity.

FIG. 9 provides a schematic of a microcapillary array with yeast cells displaying various enzyme variants and chromogenic substrate that could be cleaved by the enzyme variants and which when cleaved converts to a product that absorbs at a given wavelength and representative images.

FIG. 10 provides a plot of the transmitted light and absorbance values from 4000 capillaries were quantified.

FIG. 11 provides images showing four high absorbance variants were highlighted and further examined and shown in FIG. 11. Capillary 3 contained a bubble (a false positive) is not shown.

FIG. 12 provides date showing a bright-field (transmittance) and fluorescence images of the same array.

FIG. 13 provides correlation data between the bright-field and fluorescence measurements from the images shown in FIG. 11. More cells resulted in higher absorbance and lower bright-field intensity; more cells also resulted in higher fluorescence signal.

FIG. 14 provides data showing growth measurements, based on fluorescence signal, comparing wild-type growth, high growth, and low growth.

FIG. 15 provides data showing time course of enzyme plus substrate in dynamic assays. Serial imaging and kinetic parameters can be determined on millions of protein variants.

FIG. 16 provides data showing enzyme library screening. The platform allows for ˜500,000 enzyme variants to be screened per hour.

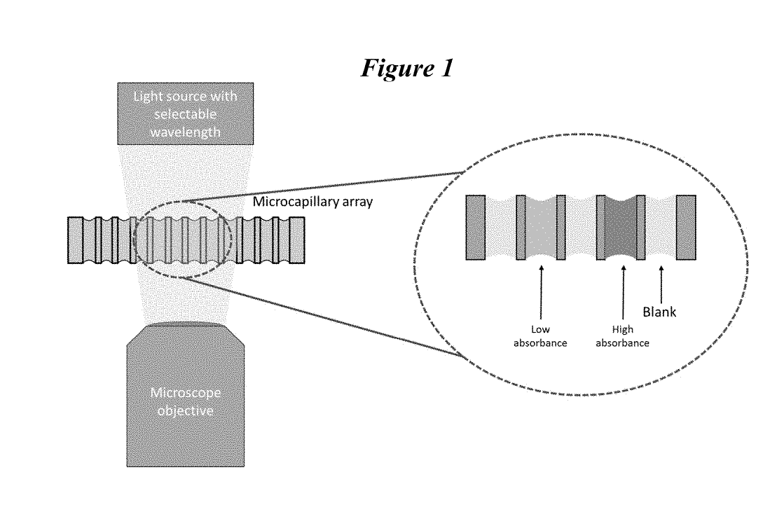

### DETAILED DESCRIPTION OF THE INVENTION

### I. Introduction

Microcapillary arrays have recently been employed in approaches for high-throughput analysis and protein engineering with large numbers of biological samples, for example in an approach that has been termed “microcapillary single-cell analysis and laser extraction” or “μSCALE”. See Chen et al. (2016) Nature Chem. Biol. 12:76-8. This approach relies on the spatial segregation of single cells within a microcapillary array, and thus enables repeated imaging, cell growth, and protein expression of the separate samples within each microcapillary of the microcapillary array. Accordingly, the technique enables massively parallel, quantitative biochemical and biophysical measurements on millions or multi-millions of samples within a microcapillary array, for example, in the analysis of millions or multi-millions of protein variants expressed from yeast, bacteria, or other suitable cells distributed throughout the array. Advantageously, the approach has allowed the simultaneous time-resolved kinetic analysis of the multiplexed samples, as well as the sorting of those cells based on targeted phenotypic features.

The development of SCALE methods and apparatus for the quantitative biochemical and biophysical analysis of populations of biological variants has also been reported in U.S. Patent Application Publication No. 2016/0244749 A1, which is incorporated by reference herein in its entirety. Extraction of the contents of a desired microcapillary according to the μSCALE approach requires, however, the inclusion of a radiation-absorbing material in each sample and the directing of electromagnetic radiation from a pulsed laser into this material, thus adding complexity to the extraction methods. In addition, earlier methods of screening of biological variants in arrays of microcavities relied on the addition of microparticles to the arrayed samples to partially or completely inhibit the transmission of electromagnetic radiation into and out of the sample in order to minimize signal emitted from microcavities lacking a desired binding activity. See U.S. Patent Application Publication No. U.S. 2014/0011690 A1. In some aspects of the invention, the screening methods do not rely on these additional sample components or manipulations, thus simplifying and improving the efficiency of the screening techniques.

As stated above, the present disclosure provides a method for high-throughput absorbance measurements of samples in microcavity arrays, including microcapillary arrays. As part of the disclosure, methods for measuring the amount of absorbance of a sample in a microcapillary are provided. Equations for determining absorbance have been described, as outlined below.

Absorbance can be defined by the following equation:

A=−log₁₀T.

T is transmittance, which can be defined as the fraction of initial light that passes through a sample:

$$T = \frac{{Intensity}_{sample}}{{Intensity}_{blank}} .$$

T is transmittance, which can be defined by the following equation:

$$T = \frac{{Intensity}_{sample}}{{Intensity}_{average}}$$

Absorbance can also be defined by the following equation:

A=2−log₁₀T%.

T is transmittance, which can be defined as the fraction of initial light that passes through a sample:

$$T = \frac{{Intensity}_{sample}}{{Intensity}_{average}} .$$

T is transmittance, which can be defined as the fraction of initial light that passes through a sample and can be expressed as a percentage:

$$T \% = \frac{{Instensity}_{sample}}{{Intensity}_{average}} * 100.$$

According to the method of the present invention, different samples are contained within a microcapillary array, where each microcapillary contains one sample. An overhead light source transmits light of a specific wavelength through the samples held in the microcapillary array. The samples in the array will absorb differing amounts of the light (depending on the concentration of the sample). The remaining light will pass through the array into the microscope objective to the detector. The differing amounts of transmitted light can be used to discriminate and characterize samples.

These methods can find use in many applications, including enzyme engineering, ELISA assays, and cell growth.

### II. Definitions

Terms used in the claims and specification are defined as set forth below unless otherwise specified. In the case of direct conflict with a term used in a parent provisional patent application, the term used in the instant specification shall control.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid. Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions,” can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “protein”, as used herein, refers both to full-length proteins or polypeptide sequences and to fragments thereof. Such fragments may include fragments that retain a functional activity, such as, for example, a binding activity. The terms “protein” and “polypeptide” are used interchangeably throughout the disclosure and include chains of amino acids covalently linked through peptide bonds, where each amino acid in the polypeptide may be referred to as an “amino acid residue”. Use of the terms “protein” or “polypeptide” should not be considered limited to any particular length of polypeptide, e.g., any particular number of amino acid residues. The subject proteins may include proteins having non-peptidic modifications, such as post-translational modifications, including glycosylation, acetylation, phosphorylation, sulfation, or the like, or other chemical modifications, such as alkylation, acetylation, esterification, PEGylation, or the like. Additional modifications, such as the inclusion of non-natural amino acids within a polypeptide sequence or non-peptide bonds between amino acid residues should also be considered within the scope of the definition of the term “protein” or “polypeptide”.

In some embodiments, the population of variant proteins is a population of proteins having minor variations, for example a population of proteins where each protein has a slightly different amino acid sequence or a different post-translational modification. In some embodiments, the variant proteins can differ by 1, 2, 3, 4, 5, 6, 7, 8, 9, or 10 or more amino acids. In some embodiments, the variants differ by at least 1 amino acid. The screening assays can, therefore, identify variant protein sequences having desirable properties. Because the screens can be performed in such large numbers at microscopic scale, huge numbers of variant proteins can be assayed in relatively short times.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., Biol. Chem. 260:2605-2608, 1985; and Cassol et al., 1992; Rossolini et al., Mol. Cell. Probes 8:91-98, 1994). For arginine and leucine, modifications at the second base can also be conservative. The term nucleic acid is used interchangeably with gene, cDNA, and mRNA encoded by a gene. Polynucleotides used herein can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

“Microcavity” and variations thereof refer to a microcavity array comprising a plurality of microcavities, each microcavity comprising a sample component, including but not limited to proteins, polypeptides, nucleic acids, small molecules, and/or cells. The term microcavity includes microcapillaries and/or microwells.

### III. High-Throughput Imaging Systems

In some embodiments, the present disclosure provides an imaging system for detecting transmittance in order to calculate the absorbance. Such imaging systems comprise a variety of components, including (A) a light source unit for providing one or more illumination and/or excitation lights to a target, the light source unit comprising at least one light source, (B) a detection unit comprising at least one detector capable of detecting transmitted light and/or fluorescence from the a sample (i.e., target) contained in a microcavity, (C) an optical train for directing the one or more illumination and/or excitation lights from the light source unit to the sample and for directing the transmitted light and/or fluorescence from the target to the detection unit; and (D) a control unit for controlling the light source unit and the detection unit. As one of skill in the art would appreciate, the components described herein are operatively connected to one another.

Any suitable microscope or imaging system capable of emitting light and detecting transmitted light for a large number of samples can be employed with the present high-throughput methods. For example, the microscope systems described in, for example, U.S. Application No. 62/433,210, can be employed with the present methods.

Such microscope or imaging systems suitable for use with the methods of the present invention include a light source with a selectable wavelength and a detector for detecting the amount of light transmitted through a given sample.

In some embodiments, the imaging systems consist of four major components, a light source, a lens, detector, and computer or other system controller. In some embodiments, other than the computer or other system controller, these components have been optimized for high-throughput applications.

In some embodiments, the imaging system comprises a light source with a selectable wavelength. In some embodiments, the light source is a plasma light source. In some embodiments, the light source is a high power plasma light source. In some embodiments, the light source is coupled to a monochromator. In some embodiments, the light source is a diode laser with a defined wavelength. In some embodiments, the laser is a UV laser. In some embodiments, the UV laser is a 375 nm laser. In some embodiments, the laser is a visible spectrum laser. In some embodiments, the visible spectrum laser is selected from the group consisting of 404 nm, 405 nm, 406 nm, 450 nm, 462 nm, 473 nm, 488 nm, 520 nm, 532 nm, 633 nm, 635 nm, 637 nm, 638 nm, 639 nm, 640 nm, 642 nm, 650 nm, 658 nm, 660 nm, 670 nm, 685 nm, and 690 nm. In some embodiments, the laser is a commercially available laser.

In some embodiments, the commercial laser includes, for example, those available from Thorlabs (see, for example, those listed on the World Wide Web at Thorlabs.com/newgrouppage9.cfm?objectgroup_id=7). In some embodiments, LEDs (light emitting diodes) are used for the light source. LEDs can provide fast on and off times, well defined emission spectra and exceptional short and long-term stability. In some embodiments, the first light engine in the plurality of light engines emits a white light. In some embodiments, the LED chips emit white light. In some embodiments, the first light engine in the plurality of light engines comprises LEDs. Such LED chips for use in the imaging systems of the present invention are readily available from commercial sources in a variety of wavelengths/colors.

In some embodiments, the imaging system consists of one or more high-resolution cameras. In some embodiments the camera is a black and white camera. In some embodiments, the camera is a color camera. In some embodiments, the imaging system consists of one real-time, high-resolution camera, one color camera. In some embodiments, the imaging system consists of one color camera and one monochrome camera, in order to expand the range of detection.

In some embodiments, while the two cameras see exactly the same field of view, they capture different information. The color camera captures RGB light while the monochrome (black and white) camera captures transmitted light in the same field. In some embodiments, to capture two different images one can employ a high-speed pulsed light source that is synchronized with the image capturing process. In some embodiments, to capture two different images one can employ a high-speed pulsed light source in combination with two cameras.

Photodiodes are traditionally used in absorbance measurements. In some embodiments, the transmitted light is detected using a photodiode. Photodiodes typically have a higher dynamic range but need be coupled with imaging of the location of each well. In some embodiments, the detector is a photodiode and the location of each well is determined. In some embodiments, a camera takes an image of the entire microcavity field. In some embodiments, the images are used to find the location of the microcavities. In some embodiments, the image of the microcavity field is then compared to the transmittance image in order to determine the transmittance for each microcavity. In some embodiments, the transmittance values are mapped to locations on the image generated of the entire microcapillary field. In some embodiments, the absorbance values are then determined for each location in the microcavity field via the photodiode. In some embodiments, the absorbance value is determined for one location in the microcavity field via the photodiode.

An optical train, also referred to as an optical assembly, is an arrangement of lenses employed as part of an imaging system and which functions to guide a light source, including a laser. The position and angle of lenses may be adjusted to guide a laser through the path required and such adjustments would be within the level of skill of one of skill in the art to adjust as needed for an imaging system. In some embodiments, the imaging system includes an optical train for directing the one or more illumination and/or excitation lights from the light source unit to the sample and for directing the transmitted light and/or fluorescence from the sample to the detection unit.

In some embodiments, the optical train for the instrument is based on a modified microscope. In some embodiments, the microscope provides front-end image collection and optical zoom with high light collection efficiency. In some embodiments, the imaging system includes a color camera. In some embodiments, the imaging system includes a black and white camera. In some embodiments, the imaging system includes a color camera and a black and white camera. In some embodiments, the optical train is coupled to one or more emission filters optimized for a particular wavelength, fluorophore, and/or ratiometric dye.

In some embodiments, the imaging system comprises 1, 2, 3, 4, 5, or 6 filters. In some embodiments, the imaging system comprises 1 filter. In some embodiments, the imaging system comprises 2 filters. In some embodiments, the imaging system comprises 3 filters. In some embodiments, the imaging system comprises 4 filters. In some embodiments, the imaging system comprises 5 filters. In some embodiments, the imaging system comprises 6 filters. In some embodiments, the one or more filters are operably coupled to the imaging system. In some embodiments, the filters are included in a filter wheel. In some embodiments, the filter wheel is operably coupled to the imaging system. In some embodiments, one filter is employed at a time, but the system is capable of switching between 1, 2, 3, 4, 5, or 6 filters in order to measure the absorbances of the samples at different wavelengths. In some embodiments, multiple filters could be used in series to narrow and/or define the range of light that reaches the sample and/or detector. In some embodiments, the one or more filters are between the light source and the sample. In some embodiments, the one or more filters are between the sample and the detector. In some embodiments, the one or more filters are in series between the sample and the detector.

In some embodiments, a uniform distribution of excitation light across the specimen plane is achieved for proper sample illumination and transmittance measurements. In some embodiments, high uniformity is achieved by using a liquid light guide. In some embodiments, a liquid light guide is employed. In some embodiments, the light passes through the liquid light guide prior to passing through the sample. In some embodiments, optics that collimate light are employed (i.e., make the rays of light accurately parallel). In some embodiments, a light guide and optics that collimate light are employed. In some embodiments, uniformity of greater than 80%, 85%, 90%, 95% or 99% can be achieved at the specimen plane. In some embodiments, uniformity of greater than 95% can be achieved at the specimen plane.

In some embodiments, one or more detectors are employed to detect a variety of different light from a variety of light sources as described herein. In some embodiments, a detection unit comprises at least a first detector that detects the transmittance of the light through the sample. In some embodiments, the detection unit comprises at least one detector that detects a fluorescence emitted from a sample. In some embodiments, the detection unit comprises at least one detector that detects other light emitted from a sample, such light from chemiluminescence (also referred to as “chemoluminescence”). In some embodiments, a detector comprises two or more detectors. In some embodiments, the two or more detectors are of differing types. In some embodiments, the two or more detectors are selected from detectors capable of detecting light transmitted through a sample, fluorescent light emitted from a sample, and/or other light emitted from a sample. In some embodiments, such light is from chemiluminescence (also referred to as “chemoluminescence”), as well as combinations thereof.

In some embodiments, the detector comprises a color camera. In some embodiments, the detector comprises a monochrome camera. In some embodiments, the detector comprises a photodiode. In some embodiments, there are two or more detectors. In some embodiments, the two or more detectors comprise a color camera, a monochrome camera, and/or a photodiode, as well as combinations thereof.

In some embodiments, the camera employs a charge-coupled device (CCD), a Complementary metal-oxide-semiconductor (CMOS), or a Scientific CMOS (sCMOS) sensor. In some embodiments, the camera employs a charge-coupled device (CCD). In some embodiments, the camera employs a Complementary metal-oxide-semiconductor (CMOS). In some embodiments, the camera employs a Scientific CMOS (sCMOS) sensor.

In some embodiments, the allowable display modes vary depending upon the type of light detected. For example, in some embodiments the images can be displayed in one of three modes: 1) full color image only (color camera), 2) fluorescent image (monochrome camera), and/or 3) full color image overlaid with fluorescent image (color camera and monochrome camera). In some embodiments, the image is displayed as a full color image only (color camera). In some embodiments, the image is displayed as a fluorescent image (monochrome camera). In some embodiments, the image is displayed as a full color image overlaid with fluorescent image (color camera and monochrome camera).

In some embodiments, the imaging system further comprises a display unit for displaying an image and/or displaying a plurality of images. A display unit can include but is not limited to a monitor, television, computer screen/terminal, LCD display, LED display or any other display unit on which an image can be viewed and with can be connected to the imaging system described herein. In some embodiments, the display unit displays the plurality of images by displaying the transmitted light image in real-time. In some embodiments, different images from different light sources can be overlaid in order to obtain additional information about a sample. In some embodiments, transmitted light, fluorescence light, and/or other light images can be combined and overlaid, depending upon the sample identity.

In some embodiments, the light source unit is controlled by the control unit such that only one light source is energized at a time.

In some embodiments, the light source unit is controlled by the control unit such that each light source is energized sequentially.

In some embodiments, the control unit synchronizes the light source unit and the detection unit. In some embodiments, the control unit synchronizes the light source unit and the detection unit such that each light source is energized sequentially.

In some embodiments, the control unit and the display unit are embedded in a computer. In some embodiments, the control unit and the display unit are part of a computer system or other controller system.

In some embodiments, the control unit is capable of performing the imaging algorithms described herein. In some embodiments, the computer is pre-programmed to run the imaging algorithms. In some embodiments, the control unit is capable of controlling the light source unit and the detection unit. In some embodiments, optionally the control unit is capable of a) comparing the light transmittance intensity obtained for each individual sample in step ii to the light transmittance intensity for a control sample; and b) calculating the absorbance of each individual sample in the array based on the comparison in order to determine differences between said samples.

One of skill in the microscopic arts would understand how to connect the various components and equipment described herein in order to employ the methods of the present invention.

### IV. Microcavity Array

In these methods, the microcavity arrays comprise any array which comprises individual chambers and which allows for the transmission of light through the array and onto a detector. In some embodiments, the arrays are microcapillary arrays. In some embodiments the microcapillary arrays comprise a plurality of longitudinally fused capillaries, for example fused silica capillaries, although any other suitable material may be utilized in the arrays. See, e.g., the arrays described U.S. Application No. 62/433,210, filed Dec. 12, 2016, U.S. application Ser. No. 15/376,588, filed on Dec. 12, 2016, PCT International Patent Publication Nos. WO 2012/007537 and WO 2014/008056, the disclosures all of which are incorporated by reference herein in their entireties.

Such arrays can be fabricated, for example, by bundling millions or billions of silica capillaries and fusing them together through a thermal process, although other suitable methods of fabrication may also be employed. The fusing process may comprise, for example, the steps of i) heating a capillary single draw glass that is drawn under tension into a single clad fiber; ii) creating a capillary multi draw single capillary from the single draw glass by bundling, heating, and drawing; iii) creating a multi-draw multi-capillary from the multi-draw single capillary by additional bundling, heating, and drawing; iv) creating a block assembly of drawn glass from the multi-multi-draw multi-capillary by stacking in a pressing block; v) creating a block pressing block from the block assembly by treating with heat and pressure; and vi) creating a block forming block by cutting the block pressing block at a precise length (e.g., 1 mm).

In some embodiments, the fabrication method further comprises slicing the silica capillaries, thereby forming very high-density glass microcapillary arrays. In some embodiments, the microcapillary arrays may be cut to approximately 1 millimeter in height, but even shorter microcapillary arrays are contemplated, including arrays of 10 m in height or even shorter. In some embodiments, even longer microcapillary arrays are contemplated, including arrays of 10 mm or even longer.

Such processes form very high-density microcapillary arrays that are suitable for use in the present methods. In an exemplary array, each microcapillary has an approximate 5 μm diameter and approximately 66% open space (i.e., representing the lumen of each microcapillary). In some arrays, the proportion of the array that is open ranges from between about 50% and about 90%, for example about 60% to about 75%, such as a microcapillary array provided by Hamamatsu that has an open area of about 67%. In one particular example, a 10×10 cm array having 5 μm diameter microcapillaries and approximately 66% open space has about 330 million total microcapillaries.

In various embodiments, the internal diameter of each microcapillary in the array ranges from between approximately 1 m and 500 m. In some arrays, each microcapillary can have an internal diameter in the range between approximately 1 m and 300 m; optionally between approximately 1 m and 100 m; further optionally between approximately 1 μm and 75 μm; still further optionally between approximately 1 μm and 50 m; and still further optionally between approximately 5 μm and 50 μm.

In some microcapillary arrays, the open area of the array comprises up to 90% of the open area (OA), so that, when the pore diameter varies between 1 μm and 500 μm, the number of microcapillaries per cm of the array varies between approximately 460 and over 11 million. In some microcapillary arrays, the open area of the array comprises about 67% of the open area, so that, when the pore size varies between 1 μm and 500 μm, the number of microcapillaries per square cm of the array varies between approximately 340 and over 800,000. In some embodiments, the number of microcapillaries per square cm of the array is approximately 400; 800; 1000; 2000; 4000; 5000; 10,0000; 25,000; 50,000; 75,000; 100,000; 200,000; 300,000; 400,000; 500,000; 600,000; 700,000; 800,000; 900,000; 1,000,000 or more.

In one particular embodiment, a microcapillary array can be manufactured by bonding billions of silica capillaries and then fusing them together through a thermal process. After that slices (0.5 mm or more) are cut out to form a very high aspect ratio glass microcapillary array. Arrays are also commercially available, such as from Hamamatsu Photonics K. K. (Japan), Incom, Inc. (Massachusetts), Photonis Technologies, S.A.S. (France) Inc., and others. In some embodiments, the microcapillaries of the array are closed at one end with a solid substrate attached to the array.

The microcapillary arrays of the instant screening methods can comprise any number of microcapillaries within the array. In some embodiments, the microcapillary array comprises at least 100,000, at least 300,000, at least 1,000,000, at least 3,000,000, at least 10,000,000, or even more microcapillaries. In some embodiments, the microcapillary array comprises at least 100,000. In some embodiments, the microcapillary array comprises at least 200,000 microcapillaries. In some embodiments, the microcapillary array comprises at least 300,000 microcapillaries. In some embodiments, the microcapillary array comprises at least 400,000 microcapillaries. In some embodiments, the microcapillary array comprises at least 500,000 microcapillaries. In some embodiments, the microcapillary array comprises at least 600,000 microcapillaries. In some embodiments, the microcapillary array comprises at least 700,000 microcapillaries. In some embodiments, the microcapillary array comprises at least 800,000 microcapillaries. In some embodiments, the microcapillary array comprises at least 900,000 microcapillaries. In some embodiments, the microcapillary array comprises at least 1,000,000 microcapillaries. In some embodiments, the microcapillary array comprises at least 2,000,000 microcapillaries. In some embodiments, the microcapillary array comprises at least 3,000,000 microcapillaries. In some embodiments, the microcapillary array comprises at least 4,000,000 microcapillaries. In some embodiments, the microcapillary array comprises at least 5,000,000 microcapillaries. In some embodiments, the microcapillary array comprises at least 10,000,000 microcapillaries. In some embodiments, the microcapillary array comprises at least 15,000,000 microcapillaries. In some embodiments, the microcapillary array comprises at least 20,000,000 microcapillaries. The number of microcapillaries within an array is preferably chosen in view of the size of the variant protein library to be screened.

The microcavity arrays are about 0.2 mm (200 μm) to about 1 mm thick and about 50 μm to about 200 μm in diameter. In some embodiments, the microcavity arrays are about 1.5 mm thick and about 150 μm in diameter. In some embodiments, the microcavity arrays are about 2 mm thick and about 200 μm in diameter. In some embodiments, the microcavity arrays are about 1 mm thick and about 100 μm in diameter. In some embodiments, the microcavity arrays are about 1 mm thick and about 10 μm in diameter. In some embodiments, the microcavity arrays are about 1 μm, 5 μm, and/or 10 μm in diameter. In some embodiments, the microcavity arrays are about 10 μm in diameter.

A variety of microcavity arrays can find use in the present methods. Exemplary microcavity array sizes are provided herein. In some embodiments, the microcavities within the arrays are about 50 μm to about 200 μm in diameter. In some embodiments, the microcavities within the arrays are about 75 μm to about 150 μm in diameter. In some embodiments, the microcavities within the arrays are about 75 μm to about 125 μm in diameter. In some embodiments, the microcavities within the arrays are about 75 μm to about 110 μm in diameter. In some embodiments, the microcavities within the arrays are about 80 μm to about 110 μm in diameter. In some embodiments, the microcavities within the arrays are about 75 μm to about 150 μm in diameter. In some embodiments, the microcavities within the arrays are about 80 μm, about 90 μm, about 100, or about 110 μm in diameter. In some embodiments, the microcavities within the arrays are about 100 μm in diameter

A variety of microcavity arrays can find use in the present methods. Exemplary sample volumes are provided herein. In some embodiments, the sample volume in each microcavity is less than about 500 nL. In some embodiments, the sample volume in each microcavity is about 5 nL to about 500 nL. In some embodiments, the volume in each microcavity is about 5 nL to about 400 nL. In some embodiments, the volume in each microcavity is about 5 nL to about 300 nL. In some embodiments, the volume in each microcavity is about 5 nL to about 200 nL. In some embodiments, the volume in each microcavity is about 5 nL to about 100 nL. In some embodiments, the volume in each microcavity is about 5 nL to about 90 nL. In some embodiments, the volume in each microcavity is about 5 nL to about 80 nL. In some embodiments, the volume in each microcavity is about 5 nL to about 70 nL. In some embodiments, the volume in each microcavity is about 5 nL to about 60 nL. In some embodiments, the volume in each microcavity is about 5 nL to about 50 nL. In some embodiments, the volume in each microcavity is about 5 nL to about 40 nL. In some embodiments, the volume in each microcavity is about 5 nL to about 30 nL. In some embodiments, the volume in each microcavity is about 5 nL to about 20 nL. In some embodiments, the volume in each microcavity is about 5 nL to about 10 nL. In some embodiments, the volume in each microcavity is about 5 nL to about 8 nL. In some embodiments, the volume in each microcavity is about 7 nL to about 8 nL. In some embodiments, the volume in each microcavity is about 7.8 nL. In some embodiments, the volume in each microcavity is about 70 pL to about 100 pL. In some embodiments, the volume in each microcavity is about 70 pL to about 90 pL. In some embodiments, the volume in each microcavity is about 70 pL to about 80 pL. In some embodiments, the volume in each microcavity is about 78.5 pL. In some embodiments, the volume in each microcavity is about 150 fL to about 1000 fL. In some embodiments, the volume in each microcavity is about 200 fL to about 1000 fL. In some embodiments, the volume in each microcavity is about 300 fL to about 1000 fL. In some embodiments, the volume in each microcavity is about 400 fL to about 900 fL. In some embodiments, the volume in each microcavity is about 500 fL to about 800 fL. In some embodiments, the volume in each microcavity is about 150 fL to 200 fL. In some embodiments, the volume in each microcavity is about 157 fL.

In some embodiments, each microcavity in the microcavity arrays of the instant screening methods further comprises an agent or agents to improve viability of the cellular expression system when cellular expression assays are used. Specifically, the agent or agents is included to prevent cell damage during the step of isolating the contents of the microcapillary of interest, for example by a laser pulse (see below). In preferred embodiments, the agent is methylcellulose (for example at 0.001 wt % to 10 wt %), dextran (for example at 0.5 wt % to 10 wt %), pluronic F-68 (for example at 0.01 wt % to 10 wt %), polyethylene glycol (“PEG”) (for example at 0.01 wt % to 10 wt %), polyvinyl alcohol (“PVA”) (for example at 0.01 wt % to 10 wt %), or the like. Alternatively, or in addition, each microcapillary in the microcapillary arrays of the instant screening methods can further comprise a growth additive, such as, for example, 50% conditioned growth media, 25% standard growth media, or 25% serum. In some embodiments, the conditioned growth media is conditioned for 24 hours. In some embodiments, the added agent is insulin, transferrin, ethanolamine, selenium, an insulin-like growth factor, or a combination of these agents or any of the agents recited above.

It should also be understood that the concentrations of each component of the screening assay within a microcavity can be modulated as desired in an assay in order to achieve an optimal outcome. In particular, it may be desirable to modulate the concentration of proteins, polypeptides, nucleic acids, small molecules, and/or cells to achieve the desired level of association between these components. The level of association will also depend on the particular affinity between these components, wherein a higher affinity results in a higher level of association for a given concentration of the components, and a lower affinity results in a lower level of association of the components for a given concentration. Concentration of various components may likewise be modulated in order to achieve optimum levels of signal output, as would be understood by those of ordinary skill in the art.

### V. Samples and/or Library Components

Libraries that can be screened according to the present methods include any library comprising a plurality of molecules as well as mixtures and/or combinations thereof.

In some embodiments, libraries comprise samples comprising biological material. In some embodiments, the library comprises samples comprising a plurality of one or more molecules and/or cells as well as mixtures and/or combinations thereof. In some embodiments, the library comprises samples comprising a plurality of one or more proteins, polypeptides, nucleic acids, small molecules, dyes, and/or cells as well as mixtures and/or combinations thereof. In some embodiments, molecules include any molecule. In some embodiments, molecules include but are not limited to proteins, polypeptides, nucleic acids, small molecules, and/or dyes as well as mixtures and/or combinations thereof. In some embodiments, libraries comprise samples comprising biological materials that comprise polypeptides, nucleic acids, small molecules, and/or cells as well as mixtures and/or combinations thereof. In some embodiments, libraries comprise samples. In some embodiments, samples include but are not limited to biological materials that comprise polypeptides, nucleic acids, small molecules, dyes, and/or cells as well as mixtures and/or combinations thereof. In some embodiments, samples contain a least one molecules and/or cell to be screened. In some embodiments, samples contain a least one to ten molecules and/or cells to be screened as well as mixtures and/or combinations thereof. In some embodiments, samples contain a plurality of molecules and/or cells to be screened as well as mixtures and/or combinations thereof. In some embodiments, the molecule to be screened is termed a target molecule. In some embodiments, the cell to be screened is termed a target cell.

The arrays provided herein allow for screening of libraries made up of proteins, polypeptides, nucleic acid, small molecules, dyes, and/or cells as well as mixtures and/or combinations thereof. In some embodiments, the target molecule to be screened is a protein, polypeptide, nucleic acid, small molecule, dye, carbohydrate, lipid, or a combination of two or more of these target molecules. In some embodiments, the proteins and/or polypeptides are selected from the group consisting of enzymes, ligands, and receptors. For example, in some embodiments the target molecule can be a lipid-modified or glycosylated protein. In some embodiments, the target molecule is a native protein.

As described above, each capillary in the microcavity array used in the instant screening methods will contain different sample components. Such sample components can include, but are not limited to proteins, polypeptides, nucleic acids, small molecules, dyes, and/or cells (i.e., target molecules and/or target cells) as well as mixtures and/or combinations thereof. In some embodiments, the library for screening comprises the variant protein, variant polypeptide, variant nucleic acid, variant small molecule, variant dye, and/or variant cells exhibiting distinguishing characteristics. In some embodiments, the variant protein, variant polypeptide, variant nucleic acid, variant small molecule, variant dye, and/or variant cells exhibit distinguishing characteristics, such that each microcavity comprises a sample that comprises a different target molecule and/or target cell from the sample found in each of the other microcavities within the array. In some embodiments, one or more microcavities within the array comprise a sample that comprises the same target molecule and/or target cell as a sample found in at least one other microcavity within the array (e.g., as duplicates for comparison).

In some embodiments, the proteins and/or polypeptides in the library to be screened in the microcavity array can be variant proteins and/or polypeptides. Variant proteins include proteins and polypeptides which are distinguishable from one another based on at least one characteristic or feature. In some embodiments, the variant proteins and/or polypeptides exhibit different amino acid sequences, exhibit different amino acid sequence lengths, are produced/generated by different methods, exhibit different activities, exhibit different chemical modifications, and/or exhibit different post-translational modifications. In some embodiments, the variant proteins and/or polypeptides exhibit different amino acid sequences. In some embodiments, the variant proteins and/or polypeptides exhibit different amino acid sequence lengths. In some embodiments, the variant proteins and/or polypeptides are produced/generated by different methods. In some embodiments, the variant proteins and/or polypeptides exhibit different activities. In some embodiments, the variant proteins and/or polypeptides exhibit different chemical modifications. In some embodiments, the variant proteins and/or polypeptides exhibit different post-translational modifications. In some embodiments, the variant protein is one of a population of variant proteins and/or polypeptides that is being subjected to the screening method and analyzed using the microcavity arrays disclosed herein. The population of variant proteins and/or polypeptides can be any population of proteins that can be suitably distributed within a microcapillary array.

In some embodiments, the nucleic acids in the library to be screened in the microcavity array can be variant nucleic acids. Variant nucleic acids include nucleic acids which are distinguishable from one another based on at least one characteristic or feature. In some embodiments, the variant nucleic acids have different nucleotide sequences, have different nucleotide sequence lengths, have been produced/generated by different methods, have different methylation patterns, have different chemical modifications, and/or exhibit other distinguishing modifications. In some embodiments, the variant nucleic acids have different nucleotide sequences. In some embodiments, the variant nucleic acids have different nucleotide sequence lengths. In some embodiments, the variant nucleic acids have been produced/generated by different methods. In some embodiments, the variant nucleic acids have different methylation patterns. In some embodiments, the variant nucleic acids have different chemical modifications. In some embodiments, the variant nucleic acids exhibit other distinguishing modifications. In some embodiments, the nucleic acid is one of a population of variant nucleic acids that is being subjected to the screening method and analyzed using the microcavity arrays disclosed herein. The population of variant nucleic acids can be any population of nucleic acids that can be suitably distributed within a microcapillary array.

In some embodiments, the small molecules in the library to be screened in the microcavity array can be variant and/or different small molecules. Variant small molecules include small molecules which are distinguishable from one another based on at least one characteristic or feature. In some embodiments, the variant small molecules have different structures, have been produced/generated by different methods, have different chemical modifications, and/or exhibit other distinguishing different features. In some embodiments, the variant small molecules have different structures. In some embodiments, the variant small molecules have been produced/generated by different methods. In some embodiments, the variant small molecules have different chemical modifications. In some embodiments, the variant small molecules exhibit other distinguishing different features. In some embodiments, the small molecules are derivatives of one another. In some embodiments, the small molecule is one of a population of small molecules that is being subjected to the screening method and analyzed using the microcavity arrays disclosed herein. The population of small molecules can be any population of small molecules that can be suitably distributed within a microcapillary array.

In some embodiments, the cells in the library to be screened in the microcavity array can be variant cells and/or cells of varying types. Variant cells include cells which are distinguishable from one another based on at least one characteristic or feature. In some embodiments, the cells are derived from different samples, are derived from different patients, are derived from different diseases, have different chemical modifications, and/or have been genetically modified. Cells can include eukaryotic and prokaryotic cells. In some embodiments, the cells are derived from different samples. In some embodiments, the cells are derived from different patients. In some embodiments, the cells are derived from different diseases. In some embodiments, the cells have different chemical modifications. In some embodiments, the cells have been genetically modified. In some embodiments, the cells can include human cells, mammalian cells, bacterial cells, and fungal cells, including yeast cells. In some embodiments, the cells can include human cells. In some embodiments, the cells can include mammalian cells. In some embodiments, the cells can include bacterial cells. In some embodiments, the cells can include fungal cells. In some embodiments, the cells can include yeast cells In some embodiments, the cell is one of a population of cells that is being subjected to the screening method and analyzed using the microcavity arrays disclosed herein. The population of cells can be any population of cells that can be suitably distributed within a microcapillary array.

In some embodiments, the population of proteins, polypeptides, nucleic acid, and/or cells is distributed in the microcavity array so that each microcavity comprises a small number of different variant proteins, variant polypeptides, variant nucleic acid, and/or cells. In some embodiments, each microcavity comprises a single different variant protein, variant polypeptide, variant nucleic acid, and/or cell per microcavity. In some embodiments, each microcavity comprises a single different variant protein per microcavity. In some embodiments, each microcavity comprises a single different variant polypeptide per microcavity. In some embodiments, each microcavity comprises a single different variant nucleic acid per microcavity. In some embodiments, each microcavity comprises a single different cell per microcavity. The population of variant proteins, variant polypeptides, variant nucleic acid, and/or cells is chosen in combination with the other components within the composition.

In some embodiments, each microcavity in the microcavity array comprises 0 to 5 different variant proteins, variant polypeptides, variant nucleic acid, and/or cells from the population of variant proteins. In some embodiments, each microcavity in the microcavity array comprises 0 to 4, 0 to 3, 0 to 2, or even 0 to 1 different variant proteins from the population of variant proteins, variant polypeptides, variant nucleic acid, and/or cells.

Accordingly, in some embodiments, the variant proteins are soluble proteins, for example soluble proteins that are secreted by a cellular expression system. Exemplary soluble variant proteins include antibodies and antibody fragments, alternative protein scaffolds, such as disulfide-bonded peptide scaffolds, extracellular domains of cell-surface receptor proteins, receptor ligands, such as, for example, G-protein coupled receptor ligands, other peptide hormones, lectins, and the like. In some embodiments, the variant proteins screened using the instant methods do not need to be covalently attached to the cell or virus that expresses them in order to be identified following a screening assay. Isolation of the contents of the desired microcapillary, followed by propagation of the cell or virus clone responsible for expression of the desired variant protein, thereby enables the identification and characterization of that variant protein. Unlike screening assays where a variant protein of interest is displayed by fusion of the protein to a molecule on the surface of a cell or virus particle, the variant proteins identified in the instant screening methods need not be altered in any way either before or after their identification. The observed activities of the variant proteins in the screens are thus more likely to represent the actual activities of those proteins in their subsequent applications. Not needing to alter variant proteins or polypeptides prior to screening also allows for more efficient screening, saving costs and time for library preparation.

In some embodiments, the variant proteins to be screened are membrane-associated proteins, for example proteins typically associated with the surface of a cell or a viral particle in an expression system. Screening of cell-associated variant proteins may be desirable where the variant protein and its target molecule mediate interactions between two cells within a biological tissue. The ability to screen cell-associated variant proteins may also be desirable in screening for interactions with traditionally “non-druggable” protein targets, such as, for example, G-protein coupled receptors or ion channels. Again, not needing to alter variant proteins or polypeptides prior to screening also allows for more efficient screening, saving costs and time for library preparation.

In some embodiments, the variant nucleic acids to be screened include any nucleic acid or polynucleotide, including nucleic acids or polynucleotides that bind to or interact with proteins. Again, not needing to alter the nucleic acids or polynucleotides prior to screening also allows for more efficient screening, saving costs and time for library preparation.

In some embodiments, the protein to be screened is an antibody, antibody fragment, such as an Fc, or an antibody fusion, including for example Fc fusions. In some embodiments, the antibody or antibody fragment can be labeled.

In some embodiments, the method employs the use of an antibody to bind to the target molecule to be screened. In some embodiments, the antibody is a labeled primary antibody or a labeled secondary antibody as is used to bind to the target molecules. A primary antibody is typically considered to be an antibody that binds directly to an antigen of interest, whereas a secondary antibody is typically considered to be an antibody that binds to a constant region on a primary antibody for purposes of labeling the primary antibody. Accordingly, secondary antibodies are frequently labeled with fluorophores or other detectable labels or are labeled with enzymes that are capable of generating detectable signals. They are generally specific for a primary antibody from a different species. For example, a goat or other animal species may be used to generate secondary antibodies against a mouse, chicken, rabbit, or nearly any primary antibody other than an antibody from that animal species, as is understood by those of ordinary skill in the art. In some embodiments, the labeled antibody is a primary or secondary antibody. In some embodiments, the labeled antibody is a fluorescent antibody or an enzyme-linked antibody.

As would be understood by those of ordinary skill in the art, when a fluorescent antibody, for example is used in the instant screening methods, the signal emitted by any excess reporter element remaining free in solution (i.e., either not bound to a variant protein or bound to a variant protein that is not bound to a target molecule) within the microcavity should not be so high that it overwhelms the signal of reporter elements associated with a target molecule via a variant protein (see, e.g., the unassociated fluorescent antibodies). Such background signals can be minimized, however, by limiting the concentration of labeled antibody or other reporter element within the microcapillary solution. In addition, where signals from the screening methods are measured using a fluorescent microscope, configuring the microscope to image a relatively narrow depth of field bracketing the location of the target molecules (e.g., the bottom of the microcapillaries when target cells have settled there by gravitational sedimentation) can minimize the background signal from reporter elements not associated with the target molecule.

### VI. Libraries

The number of microcapillaries within an array is generally chosen in view of the size of the library to be screened. In some embodiments, the library size is at least 100,000, at least 300,000, at least 1,000,000, at least 3,000,000, at least 10,000,000, or even more proteins, polypeptides, nucleic acids, small molecules, and/or cells as well as mixtures and/or combinations thereof. In some embodiments, the library comprises at least 100,000 proteins, polypeptides, nucleic acids, small molecules, and/or cells as well as mixtures and/or combinations thereof. In some embodiments, the library comprises at least 200,000 proteins, polypeptides, nucleic acids, small molecules, and/or cells as well as mixtures and/or combinations thereof. In some embodiments, the library comprises at least 300,000 proteins, polypeptides, nucleic acids, small molecules, and/or cells as well as mixtures and/or combinations thereof. In some embodiments, the library array comprises at least 400,000 proteins, polypeptides, nucleic acids, small molecules, and/or cells as well as mixtures and/or combinations thereof. In some embodiments, the library comprises at least 500,000 proteins, polypeptides, nucleic acids, small molecules, and/or cells as well as mixtures and/or combinations thereof. In some embodiments, the library comprises at least 600,000 proteins, polypeptides, nucleic acids, small molecules, and/or cells as well as mixtures and/or combinations thereof. In some embodiments, the library comprises at least 700,000 proteins, polypeptides, nucleic acids, small molecules, and/or cells as well as mixtures and/or combinations thereof. In some embodiments, the library comprises at least 800,000 proteins, polypeptides, nucleic acids, small molecules, and/or cells as well as mixtures and/or combinations thereof. In some embodiments, the library comprises at least 900,000 proteins, polypeptides, nucleic acids, small molecules, and/or cells as well as mixtures and/or combinations thereof. In some embodiments, the library comprises at least 1,000,000 proteins, polypeptides, nucleic acids, small molecules, and/or cells as well as mixtures and/or combinations thereof. In some embodiments, the library comprises at least 2,000,000 proteins, polypeptides, nucleic acids, small molecules, and/or cells as well as mixtures and/or combinations thereof. In some embodiments, the library comprises at least 3,000,000 proteins, polypeptides, nucleic acids, small molecules, and/or cells as well as mixtures and/or combinations thereof. In some embodiments, the library comprises at least 4,000,000 proteins, polypeptides, nucleic acids, small molecules, and/or cells as well as mixtures and/or combinations thereof. In some embodiments, the library comprises at least 5,000,000 proteins, polypeptides, nucleic acids, small molecules, and/or cells as well as mixtures and/or combinations thereof. In some embodiments, the library comprises at least 10,000,000 proteins, polypeptides, nucleic acids, small molecules, and/or cells as well as mixtures and/or combinations thereof. In some embodiments, the library comprises at least 15,000,000 proteins, polypeptides, nucleic acids, small molecules, and/or cells as well as mixtures and/or combinations thereof. In some embodiments, the library comprises at least 20,000,000 proteins, polypeptides, nucleic acids, small molecules, and/or cells as well as mixtures and/or combinations thereof. In some embodiments, the library comprises at least 22,000,000 proteins, polypeptides, nucleic acids, small molecules, and/or cells as well as mixtures and/or combinations thereof. In some embodiments, the library comprises at least 25,000,000 proteins, polypeptides, nucleic acids, small molecules, and/or cells as well as mixtures and/or combinations thereof. In some embodiments, the library comprises at least 50,000,000 proteins, polypeptides, nucleic acids, small molecules, and/or cells as well as mixtures and/or combinations thereof. In some embodiments, the library comprises at least 75,000,000 proteins, polypeptides, nucleic acids, small molecules, and/or cells as well as mixtures and/or combinations thereof. In some embodiments, the library comprises at least 100,000,000 proteins, polypeptides, nucleic acids, small molecules, and/or cells as well as mixtures and/or combinations thereof.

It would be understood by one of skill in the art that each microcavity will typically comprise many multiple copies of the same protein, polypeptide, nucleic acid, small molecule, and/or cell, depending on the source and expression level of the particular protein, polypeptide, nucleic acid, small molecule, and/or cell as well as mixtures and/or combinations thereof. In some embodiments, each microcavity will comprise thousands, tens of thousands, hundreds of thousands, millions, billions, or even more molecules of a particular protein, polypeptide, nucleic acid, small molecule, and/or cell, depending on how the protein, polypeptide, nucleic acid, small molecule, and/or cell is delivered to or expressed within the microcavity as well as mixtures and/or combinations thereof. In some embodiments, one, two, three, four, or more types of protein, polypeptide, nucleic acid, small molecule, and/or cell can be in a sample and/or in the microcavity.

The population of proteins, polypeptides, nucleic acids, and/or small molecules, as well as mixtures and/or combinations thereof, is typically generated using a genetic library in a biological expression system, for example in an in vitro (e.g., cell-free) expression system or in an in vivo or cellular expression system. The population of proteins, polypeptides, nucleic acids, and/or small molecules, as well as mixtures and/or combinations thereof, can also be generated via any known synthesis methods. Exemplary cellular expression systems include, for example, animal systems (e.g., mammalian systems), fungal systems (e.g., yeast systems), bacterial systems, insect systems, or plant systems. In some embodiments, the expression system is a mammalian system or a yeast system. The expression system, whether cellular or cell-free, typically comprises a library of genetic material encoding the population of variant proteins. Cellular expression systems offer the advantage that cells with a desirable phenotype, for example cells that express a particular variant protein of interest, such as a variant protein capable of associating with an immobilized target molecule with high affinity, can be grown and multiplied, thus facilitating and simplifying the identification and characterization of the proteins of interest expressed by the cells.

Genetic libraries encoding large populations of proteins, polypeptides, nucleic acids, and/or small molecules, as well as mixtures and/or combinations thereof, are well known in the art of bioengineering. Such libraries are often utilized in systems relying on the process of directed evolution to identify proteins with advantageous properties, such as high-affinity binding to target molecules, stability, high expression, or particular spectroscopic, e.g., fluorescence, or enzymatic activities. Often the libraries include genetic fusions with sequences from the host expression system, for example fragments of proteins directing subcellular localization, where the expressed population of variant fusion proteins are directed by the targeting fragment to a particular location of the cell or virus particle for purposes of activity screening of the variant protein population. Large numbers of variant proteins, polypeptides, nucleic acids, small molecules, and/or cells (e.g., 10⁶ variants, 10⁸ variants, 10¹⁰ variants, 10¹² variants, or even more variants), as well as mixtures and/or combinations thereof, can be generated using routine bioengineering techniques, as is well known in the art. In some embodiments, the library is purchased from a commercial source.

### VII. High-Throughput Method for Determining Absorbance

The present invention provides for a high-throughput method for determining the absorbance for multiple samples in a microcavity array, using for example, the arrays described above. Such samples can be any samples known in the art, including those discussed above, such as proteins, polypeptides, nucleic acid, and/or cells, as well as combinations thereof. In some embodiments, the method comprises the steps of: i) transmitting light of a definable wavelength through samples contained in said microcavity array, wherein one sample is loaded into each microcavity within the array; ii) measuring the light transmitted through said samples with a detector, wherein the light transmitted is measured for each individual sample within the array in order to obtain a light transmittance intensity for each individual sample within the array; iii) comparing the light transmittance intensity obtained for each individual sample in step ii) to the light transmittance intensity for a control sample; and iv) calculating the absorbance of each individual sample in the array based on the comparison in step iii). In some embodiments, the method further comprises distinguishing between microcavities with different transmittance and/or absorbance characteristics. In some embodiments, microcavities with high transmittance and low absorbance are distinguished from microcavities with low transmittance and high absorbance. High and or low values can include values as compared to other microcavities within the array and/or as compared to a control sample. In some embodiments, microcavities with high transmittance and low absorbance are selected for further analysis. In some embodiments, microcavities with low transmittance and high absorbance are selected for further analysis.

In some embodiments, the method further comprises loading one sample into each microcavity prior to light transmission in step i).

In some embodiments, the measurement in step ii) occurs simultaneously for all the samples in the microcavity array. In some embodiments, the measurements are performed in real time. In some embodiments, the measurements are performed on the same samples as part of a time course. In some embodiments, the time course is on the order of seconds, minutes, and/or hours. In some embodiments, the time course is over 1 hour, 2 hours, 3, hours, 6 hours, 8 hours, 12 hours, 24 hours, 36 hours, and/or 48 hours or more.

As provided above, Absorbance refers to the ability of a sample to absorb light which is passed through the sample. Transmittance refers to the ability of a sample to transmit light which is passed through the sample and can be express as Intensityₛₐₘₚₗₑ. Transmittance and absorbance can be measured by a variety of know calculation methods.

In some embodiments, absorbance can be defined by the following equation:

A=−log₁₀T.

In some embodiments, T is transmittance which can be defined as the fraction of initial light that passes through a sample:

$$T = \frac{{Intensity}_{sample}}{{Intensity}_{control}} .$$

In some embodiments, the sample intensity (Intensityₛₐₘₚₗₑ) and the intensity of the control (Intensity_{control}) are measured by the system.

In some embodiments, T is transmittance which can be defined as the fraction of initial light that passes through a sample:

$$T = \frac{{Intensity}_{sample}}{{Intensity}_{blank}} .$$

In some embodiments, the sample intensity (Intensityₛₐₘₚₗₑ) and the intensity of the blank (Intensity_{blank}) are measured by the system.

In some embodiments, transmittance can be defined by the following equation:

$$T = \frac{{Intensity}_{sample}}{{Intensity}_{average}} .$$

In some embodiments, the sample intensity (Intensityₛₐₘₚₗₑ) and the average intensity (Intensity_{average}) are measured by the system. Average intensity (Intensity_{average}) can be determined by determining the intensity for all the samples in the microcavities within the array and calculating the average intensity of all the microcavities.

In some embodiments, absorbance can be defined by the following equation:

A=2−log₁₀T%.

In some embodiments, T is transmittance which can be defined as the fraction of initial light that passes through a sample:

$$T \% = \frac{{Intensity}_{sample}}{{Intensity}_{control}} * 100$$

In some embodiments, the sample intensity (Intensityₛₐₘₚₗₑ) and the intensity of the control (Intensity_{control}) is measured by the system.

In some embodiments, T is transmittance which can be defined as the fraction of initial light that passes through a sample:

$$T \% = \frac{{Intensity}_{sample}}{{Intensity}_{blank}} * 100.$$

In some embodiments, the sample intensity (Intensityₛₐₘₚₗₑ) and the intensity of the blank (Intensity_{blank}) is measured by the system.

In some embodiments, T is transmittance which can be defined as the fraction of initial light that passes through a sample and can be expressed as a percentage:

$$T \% = \frac{{Instensity}_{sample}}{{Intensity}_{average}} * 100.$$

In some embodiments, the sample intensity (Intensityₛₐₘₚₗₑ) and the average intensity (Intensity_{average}) are measured by the system. Average intensity (Intensity_{average}) can be determined by determining the intensity for all the samples in the microcavities within the array and calculating the average intensity of all the microcavities.

In some embodiments, the transmittance is measured and the absorbance determined for all the samples in the microcavity array. In some embodiments, the absorbance is used to determine one or more spectrometric characteristics for all the samples in the microcavity array. In some embodiments, the method further comprises using the absorbance to determine one or more spectrometric characteristics. In some embodiments, transmittance is directly measured and absorbance is calculated by the measured transmittance. In some embodiments, the absorbance allows for a determination of the spectrometric characteristics of the microcavity sample. In some embodiments, transmittance is directly measured and then absorbance calculated.

Generally, most spectrometers are linear from absorbance of 0 to 1 (100% to 10% transmittance). In some embodiments, spectrometers can measure from 0 to 2 (100% to 1% transmittance). In some embodiments, the absorbance is compared to one or more control or blank microcavities within the array. In some embodiments, high absorbance as compared to a control or blank microcavity indicates a 10%, 20%, 30%, 40%, 50%, 60%, 70%, 80%, 90%, or 100% increase or more in the absorbance as compared to the control. In some embodiments, high absorbance results from low transmittance, wherein low transmittance as compared to a control or blank microcavity indicates a 10%, 20%, 30%, 40%, 50%, 60%, 70%, 80%, 90%, or 99% decrease or more in the transmittance as compared to the control. In some embodiments, low absorbance as compared to a control or blank microcavity indicates a 10%, 20%, 30%, 40%, 50%, 60%, 70%, 80%, 90%, or 100% decrease or more in the absorbance as compared to the control. In some embodiments, low absorbance results from high transmittance, wherein high transmittance as compared to a control or blank microcavity indicates a 10%, 20%, 30%, 40%, 50%, 60%, 70%, 80%, 90%, or 99% increase or more in the transmittance as compared to the control.

In some embodiments the control is a negative control solution or a blank. In some embodiments, the control solution is the buffer and/or solution the reaction takes place in. In some embodiments, the control solution is the buffer and/or solution the sample is stored in. In some embodiments, the amount of light transmitted by the control is very high. In some embodiments, the amount of light of absorbed by the control is very low and/or minimal. In some embodiments, the amount of light passing through the control is high. In some embodiments, the amount of light passing through the control is considered 100% of the light from the light source. In some embodiments, the amount of light passing through the control is defined as the Intensity_{control}. In some embodiments, the amount of light passing through the blank is defined as the Intensity_{blank}. In some embodiments, high absorbance occurs with samples that have low transmittance compared to the control. In some embodiments, high absorbance occurs with samples that have low transmittance compared to the blank. In some embodiments, high absorbance occurs with samples that have low transmittance compared to the control and/or blank. In some embodiments, samples that have higher absorbance than the negative control are selected for further analysis.

In some embodiments, the control is a positive control solution. In some embodiments, the control solution is the buffer and/or solution the reaction takes place in. In some embodiments, the control solution is the buffer and/or solution the sample is stored in. In some embodiments, the amount of light transmitted by the control is very low. In some embodiments, the amount of light of absorbed by the control is very high and/or maximal. In some embodiments, the amount of light passing through the control is low. In some embodiments, the amount of light passing through the control is considered 0% of the light. In some embodiments, the amount of light passing through the control is defined as the Intensity_{control}. In some embodiments, the amount of light passing through the blank is defined as the Intensity_{blank}. In some embodiments, low absorbance occurs with samples that have high transmittance compared to the control. In some embodiments, low absorbance occurs with samples that have high transmittance compared to the blank. In some embodiments, low absorbance occurs with samples that have high transmittance compared to the control and/or blank. In some embodiments, samples that have lower absorbance than the positive control are selected for further analysis.

Any assay which results in a change in the light transmitted through a sample in the microcavity array or which results in the emission of light capable of being detected can be employed with the present invention. In some embodiments, both transmitted light and emitted light can be measured in order to determine different properties or characteristics for the samples in the microcavity arrays, hereinafter referred to as “spectrometric characteristics”. In some embodiments, the spectrometric characteristics are selected from the group consisting of concentration, enzyme activity, enzyme-substrate interaction, receptor-ligand binding, complex stability, protein stability/folding, cell growth, peak absorbance, antibody aggregation, protein folding, etc., including all of those discussed in detail below.

Any assay for which differences in transmittance can be detected can be employed with the present methods and the absorbance calculated for such methods. The absorbance can be used to determine a variety of spectrometric characteristics based on a variety of known assays, including for example, colorimetric assays, such as those where a chromogenic substrate is converted by enzyme into light-absorbing product. The absorbance can be used to determine the concentration of an absorbing chemical (which can be calculated via the Beer-Lambert law, as described in, for example, Ingle, J. D. J.; Crouch, S. R. (1988); Spectrochemical Analysis.) In some embodiments, chemical (small molecule) characterization can be performed based on the light transmittance. In some embodiments, the concentration of an absorbing chemical or the absorption spectrum can be determined based on the transmitted light measurements. In some embodiments, the concentration of a small molecule can be calculated via the Beer-Lambert law. In some embodiments, an absorption spectrum can be determined and a spectral fingerprint developed for small molecules analyzed, such a spectral fingerprint can be based on the absorbance versus the wavelength in order to determine unique spectral fingerprints for various small molecule chemical entities.

In some embodiments, enzyme activity is measured using any known enzyme activity assays known in the art which are capable of inducing measurable changes in light transmittance. In some embodiments, a substrate capable of emitting or absorbing light can also be employed, such that when the substrate is modified (for example, cleaved), by the enzyme light is emitted. In some embodiments, the substrate is not covalently attached to the sample component being assayed and/or analyzed.

In some embodiments, the absorbance can be used to generate an absorption spectrum for each sample in the microcavity array, where absorbance is calculated and plotted versus various wavelengths of light to determine a fingerprint for each sample in the microcavity array. This can be used to determine concentration and/or identity of the molecule within the samples in the microcavity array.

In some embodiments, any of the known colorimetric assays in which the substrate converts from one color/wavelength to another color/wavelength can be employed with the present methods.

In some embodiments, a change in peak absorbance wavelength can be measured and used to determine characteristics about the sample, including for example any of the spectrometric characteristics discussed above.

Concentration or cell growth results can be based on a densitometric assay. In some embodiments, a densitometric assay is any assay in which an increase in the number of molecule in a sample results in a decrease in transmittance or where the decrease in number of molecules results in an increase in the transmittance. In some embodiments, a densitometric assay is an assay wherein light is blocked by material in the sample. In some embodiments, the absorbance can be used to determine growth of cells contained with the samples in the microcavity array. In some embodiments, large numbers of cells (for example, due to cell growth) will block light from passing through array, resulting in reduced transmittance and high absorbance. In some embodiments, small numbers of cells (for example, due to no cell growth) will not block light from passing through array, resulting in increased transmittance and low absorbance.

Additionally, in many of the colorimetric assays, the substrate converts from one color/wavelength to another color/wavelength. As such, a change in peak absorbance wavelength would be another “spectrometric characteristic.” In some embodiments, the substrate conversion from one color/wavelength to another color/wavelength is due a property of the proteins, polypeptides, nucleic acid, and/or cells, or combinations thereof in the sample.

In some embodiments, receptor-ligand binding can be measured using a protein dye that binds the ligand, and the absorbance build up on the receptor (on a cell surface or immobilized on a bead as binding occurs) can be seen. In such embodiments, a decrease in transmittance and an increase in absorbance is indicative of receptor-ligand binding.

In some embodiments colorimetric assays include DNA-protein binding assay kit. In some embodiments, DNA-protein interactions can be analyzed using DNA-protein binding assay kits. Such kits provide colorimetric reagents capable of emitting light upon the occurrence of DNA-protein interactions. In some embodiments, labeled nucleic acids (e.g., labeled DNA) is used as a reagent which emits a colorimetric signal upon binding to a protein or polypeptide in the microcavity sample. In some embodiments, a reverse type assay is employed, wherein a labeled protein or polypeptide is used as a reagent which emits a colorimetric signal upon binding to a nucleic acid in the microcavity sample.

In some embodiments, antibody self-aggregation assays with gold nanoparticles can be employed with the present methods. In some embodiments, monodisperse gold nanoparticles can be modified to attach particles on their surface. In some embodiments, the modification allows the gold nanoparticles to interact with a particular target. In some embodiments, gold nanoparticles (AuNPs) are employed as part of a colorimetric assay. In some embodiments, AuNP aggregation results in a change in the transmittance of the sample at one wavelength as compared to another wavelength. In some embodiments, the AuNPs aggregate such that the sample absorbs more light in one wavelength than in another wavelength. Depending on the binding interactions of the attached particles, the nanoparticles may aggregate. As described in Saha et al., the aggregation of gold nanoparticles (AuNPs) of sizes that are, for example, less than 3.5 nm in diameter induce interparticle surface plasmon coupling, resulting in a visible color change from red to clue at nanomolar concentrations. In some embodiments, the color change during AuNP aggregation (due for example to redispersion of an aggregate) provides an absorption-based colorimetric assay for detecting a target analyte that directly or indirectly induces the AuNP aggregation and/or redispersion. See, for example, Saha, K., Agasti, S. S., Kim, C., Li, X. & Rotello, V. M. Gold nanoparticles in chemical and biological sensing. Chem. Rev. 112, 2739-79 (2012)).

In some embodiments, enzyme activity results are based on a colorimetric assay. In some embodiments, the colorimetric assay is an enzyme based light absorbing assay. In some embodiments, colorimetric assays include ELISAs (enzyme-linked immunosorbent assays) and or enzyme immunoassay (EIA). In some embodiments, ELISA can be employed with the present methods, wherein the light generated from the ELISA binding activity can be detected using the present methods. In some embodiments, variant enzymes could generate different specific light wavelengths. In some embodiments, the same enzyme acting on different substrates whose products absorb in different wavelengths, allowing for a determination of multiple spectrometric characteristics for each microcavity sample. In some embodiments, enzymes can be engineered with different selectivity profiles, based on the selectivity for different substrates.

In some embodiments, protein and/or polypeptide stability, including folding state, can be assessed using the present methods. Changes in transmittance between unfolded proteins and/or polypeptides as compared to folded proteins and/or polypeptides can be determined and differences in absorbance calculated in order to determine folding patterns and/or properties for the protein and/or polypeptides in the microcavity samples.

In some embodiments, receptor-ligand interactions can be assessed, as well as receptor-ligand complex stability. Changes in transmittance between bound receptor-ligand complexes and unbound receptor-ligand complexes can be determined and differences in absorbance calculated in order to determine complex stability patterns and/or properties for the receptor-ligand complexes in the microcavity samples.

Any of the above methods could be combined to for multiplex-type assays. Such multiplex methods can be employed to detect multiple characteristics in a sample, multiple molecules in sample, or any other combination of sample features.

In some embodiments, the methods can include both fluorescent and non-fluorescent detection methods. In some embodiment's, the present methods can be employed with the fluorescent labeling methods described in U.S. Application No. 62/433,210, filed Dec. 12, 2016, and/or U.S. application Ser. No. 15/376,588.

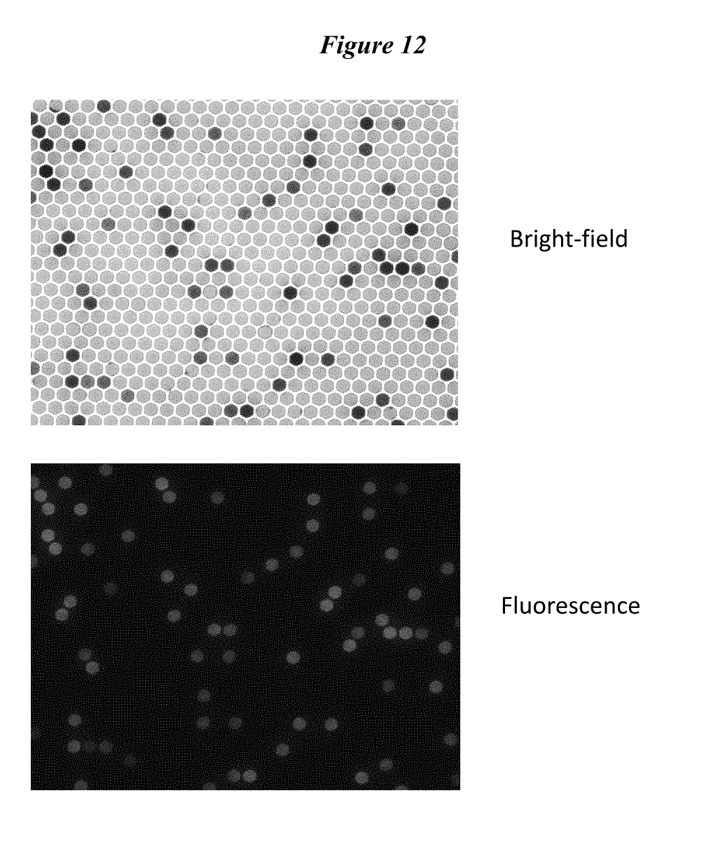

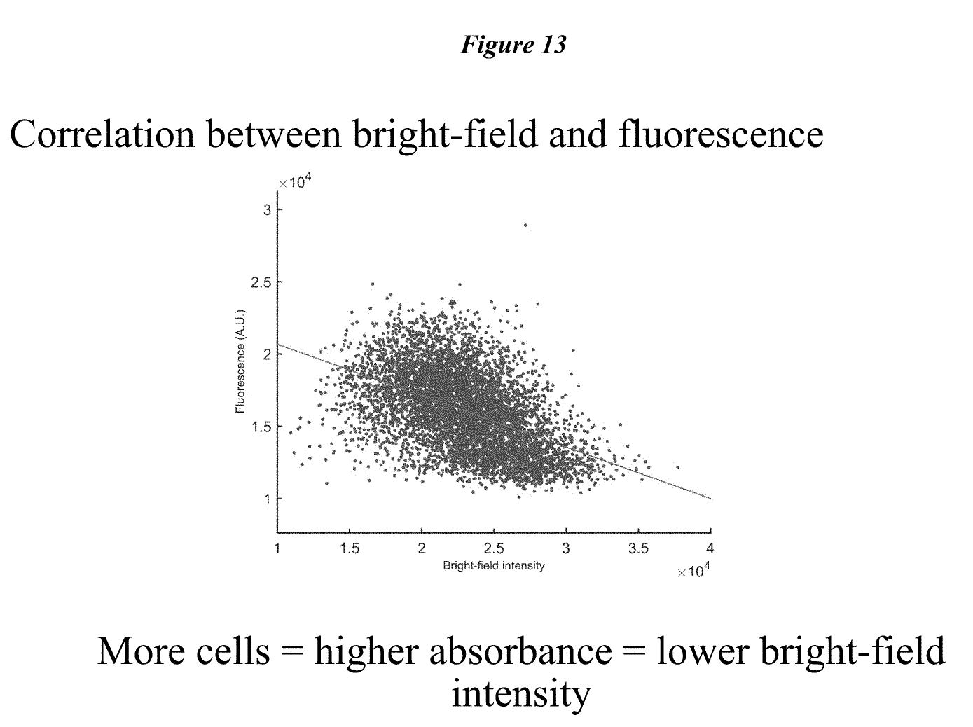

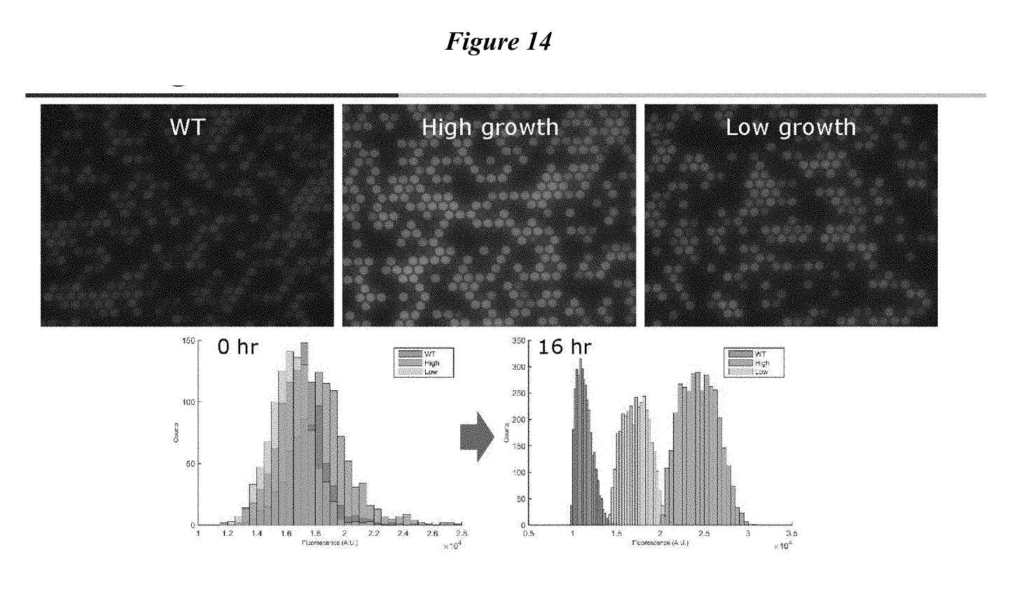

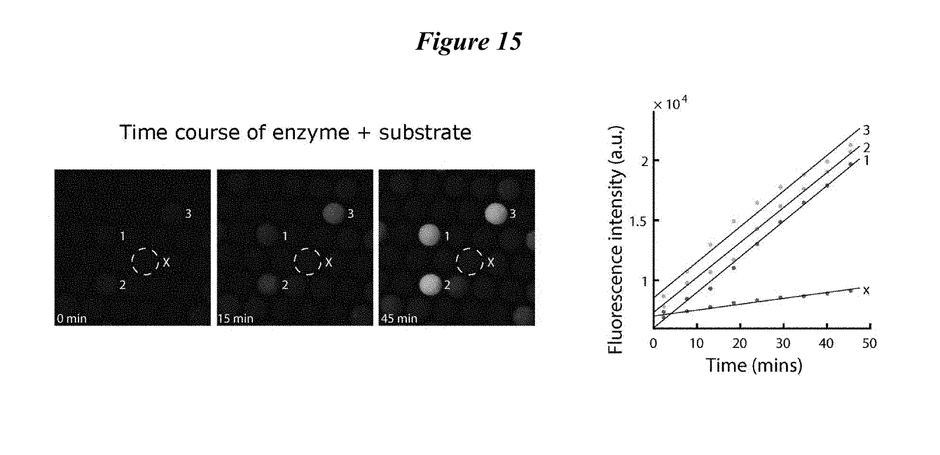

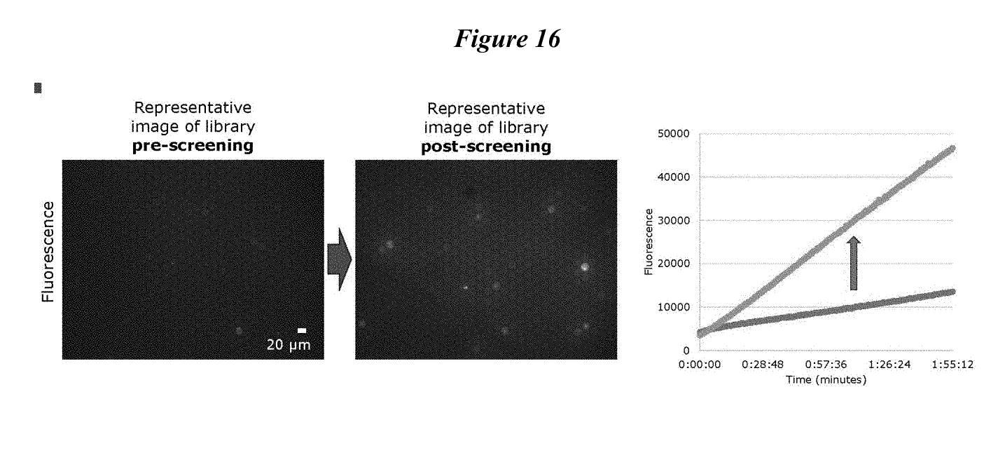

For example, both absorbance and fluorescence can be detected, as provided in FIGS. 12 and 13. In some embodiments, determining both absorbance and fluorescence can be used to validate a single metric or characteristic (as provided in FIG. 13). In some embodiments, both absorbance and fluorescence can be employed to determine one or more spectrometric characteristics and/or densitometric characteristic, as described above.

In some embodiments, fluorescence can be used for one metric or characteristic and then absorbance for another metric or characteristic. In some embodiments, fluorescence can be used for one metric or characteristic, for example how much an enzyme is expressed, and then absorbance for another metric or characteristic, for example, the enzyme variant activity. In some embodiments, a fluorescent substrate and an absorbent substrate can be employed to determine one or more spectrometric characteristics and/or densitometric characteristics, as described above. In some embodiments, absorbance can be used as a measure of cell growth, and fluorescence as the biological readout, for example for binding or enzymatic activity. In some embodiments, absorbance can be used as a measure of cell growth and fluorescence as a measure of binding or enzymatic activity.

In some embodiments, a detectable signal can be generated in connection with a binding event, such as, for example, the association of a variant protein with a detection molecule. In these embodiments, the variant protein and or detection molecule can be components in cellular pathway, such as, for example, an intracellular signaling pathway. Such a pathway should include, or be engineered to include, a detectable signal as the downstream readout of the pathway. The detectable signal in these embodiments would typically be generated inside the microcavity.

Many intracellular signaling pathways have been developed for use in high throughput screening assays, in particular in drug discovery screens, and can be adapted for use in the instant assays. See, e.g., Michelini et al. (2010) Anal. Bioanal. Chem. 398:227-38. In particular, any cellular assay where a binding event with a target molecule on the surface of a cell results in the generation of a measurable signal, in particular a fluorescent signal, can be used as a reporter element in the instant assays. In some embodiments, the cells can be engineered to express a target molecule of interest on their surface, so that the binding of a particular variant protein to the target molecule and the consequent activation of the intracellular signaling pathway result in the production of a detectable signal from the reporter element, thus enabling the identification of the microcavity as a positive hit. The expression of a green fluorescent protein (GFP), or any of a wide variety of variant fluorescent proteins, is often used as a readout in such cellular assays and can serve as the reporter element endpoint in the instant methods. Such methods could also be combined with a second absorbent and/or different fluorescent molecule in order to determine binding, enzyme activity, or any other characteristic of a molecule associated with the cell expressing the GFP. Alternatively, the signaling readout can be provided by luciferase or other related enzymes that produce bioluminescent signals, as is well understood by those of ordinary skill in the art. See, e.g., Kelkar et al. (2012) Curr. Opin. Pharmacol. 12:592-600. Other well-known enzymatic reporters from bacterial and plant systems include β-galactosidase, chloramphenicol acetyltransferase, β-glucuronidase (GUS), and the like, which can be adapted for use in the instant screening assays with suitable colorogenic substrates. Transcriptional reporters using firefly luciferase and GFP have been used extensively to study the function and regulation of transcription factors. They can likewise be adapted for use in combination with the instant screening assays. Exemplary intracellular signaling systems are available commercially, for example the Cignal™ Reporter Assay kits from Qiagen (see, e.g., www.sabiosciences.com/reporterassays.php), which are available with either luciferase or GFP readouts. Such systems can be suitably re-engineered for use in the instant screening methods.

### EXAMPLES

### Example 1

### High-Throughput Absorbance Measurements of Samples in Microcapillary Arrays

### SUMMARY

The present invention provides a method for measuring the amount of absorbance of a sample in a microcapillary. Absorbance is defined by the following equation

A=−log₁₀T

Where T is transmittance, which is defined as the fraction of initial light that passes through a sample.

$$T = \frac{{Intensity}_{sample}}{{Intensity}_{blank}}$$

In this method, different samples are contained within a mlcrocapillary array, where each microcapillary contains one sample. An overhead light source transmits light of a specific wavelength through the samples held in the microcapillary array. The samples in the array will absorb differing amounts of the light (depending on the concentration of the sample). The remaining light will pass through the array into the microscope objective to the detector. The differing amounts of transmitted light can be used to discriminate and characterize samples.

These methods can be used in many applications, including enzyme engineering, ELISA assays, and cell growth.

### Method Components:

Light source with selectable wavelength
a. In one current setup, a high power plasma light source coupled to 6 filters in a filter wheel is used.
b. Alternatively, a monochromator can be used.

Detector:
a. Camera: current setup is black and white, but a color camera can also be used (the RGB values can be used for further discrimination).
b. Photodiode: traditionally used in absorbance measurements. These have a higher dynamic range, but the image the location of each well is needed.

Absorbance readout:
a. Colorimetric assays: chromogenic substrate that is converted by enzyme into light-absorbing product.
b. Cell growth in microcapillaries: large amounts of cells will block light from passing through array.

Uses of method:
1. Characterizing enzymes/enzyme engineering.
2. Quantifying the amount of cell growth in the capillary.

### Example 2

### Absorbance Demonstration Data

### SUMMARY

To demonstrate high-throughput absorbance measurements, several features were characterized. Initial efforts focused on studying basic parameters involved in absorbance measurements: concentration and wavelength. After these efforts, the high-throughput absorbance measurements with sample containing diverse enzyme variants were demonstrated.

Throughout these studies, absorbance was measured via the following protocol:
1. The light passing through a microcapillary containing a “blank” (B) is quantified. The blank is identical to the sample but does not contain any absorbing material.
2. The light passing through a microcapillary containing a “sample” (S) is quantified.
3. Transmittance is calculated as

$$T = \frac{S}{B}$$

4. Absorbance is calculated as

$$Abs = {log}_{10} ( T ) = {log}_{10} ( \begin{matrix}S \\ B\end{matrix} )$$

Using this method, a sample that transmits 1% compared to the blank has an absorbance (OD) of 2.

### Absorbance and Concentration

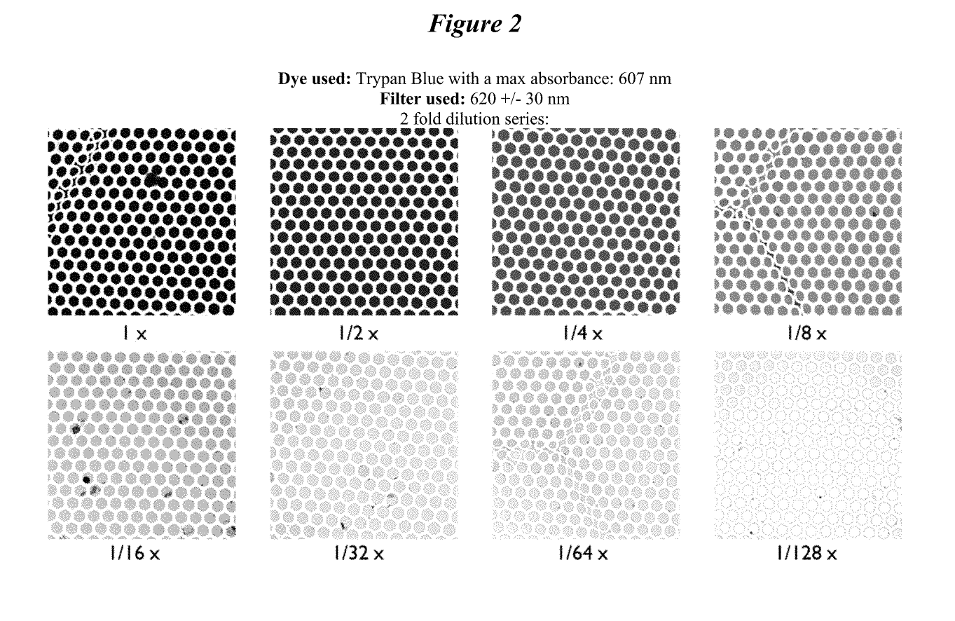

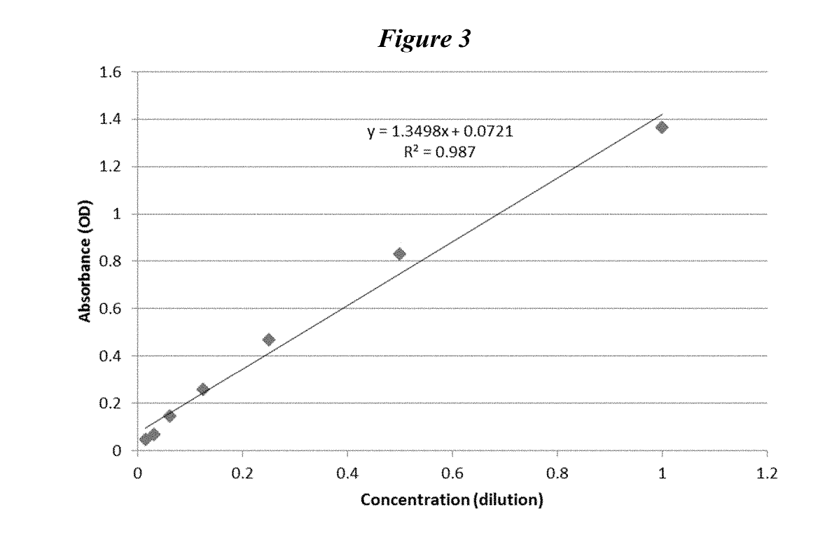

The absorbance measurements must be able to separate samples via the concentration of the absorbing material. To demonstrate this ability, a dilution series was created and the absorbance measured. For this first proof of concept assay, the dye used was Trypan Blue Trypan Blue with a max absorbance: 607 nm. The filter used was 620+/−30 nm. As shown in FIG. 2, a 2-fold dilution series was examined.

### Absorbance and Wavelength

Different materials absorb various amounts of radiation depending on wavelength. This wavelength dependent behavior can be used to characterize and identify the presence of various materials. To characterize the relationship between wavelength and absorbance, 4 sample dyes that absorbed at various wavelengths were used combined with 4 filter cubes. Using each filter cube, the 4 samples and a “blank” were imaged.

TABLE 1
Dyes

| | Max absorbance |
|---|---|
| Sample | (nm) |
| 1 | 395* |
| 2 | 430 |
| 3 | 525 |
| 4 | 630 |

*According to literature

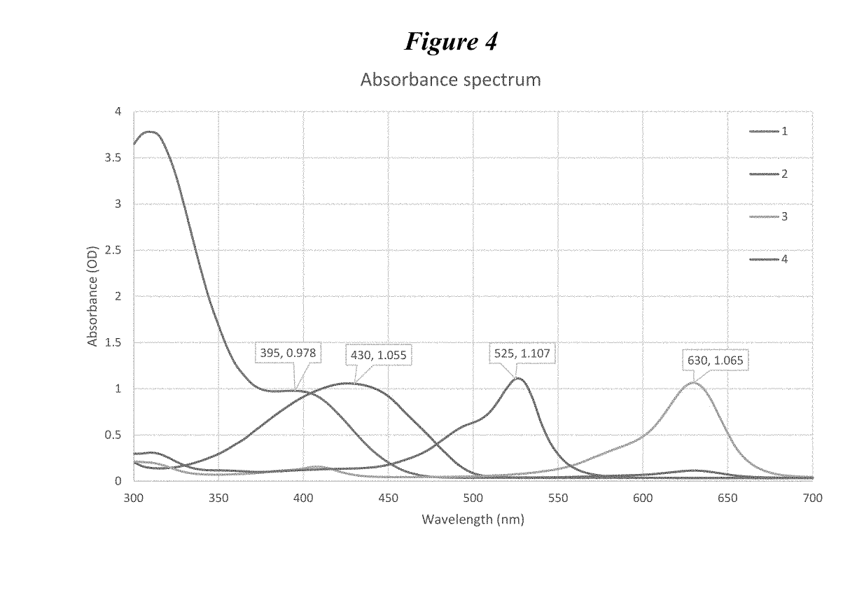

Each sample was diluted to OD 1 at their max absorbance wavelength. The data is provided in FIG. 4.

TABLE 2
Filter cubes

| Cube | Center (nm) | Width (nm) | Manufacturer |
|---|---|---|---|
| 1 | 350 | 50 | Nikon C-FL DAPI |
| | | | (excitation filter) |
| 2 | 475 | 40 | Omega |
| 3 | 525 | 45 | Omega |
| 4 | 620 | 60 | Omega |

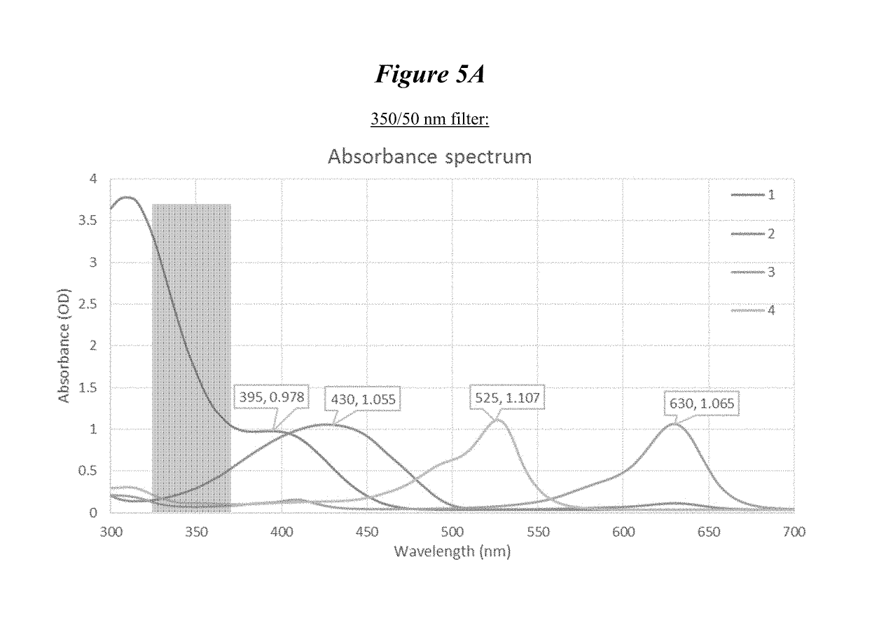

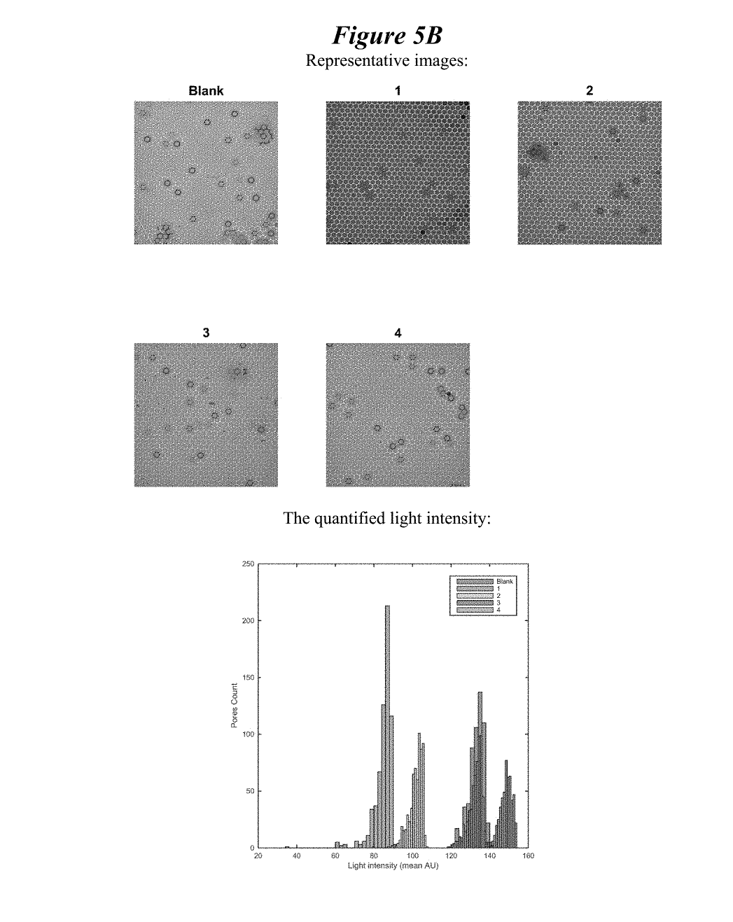

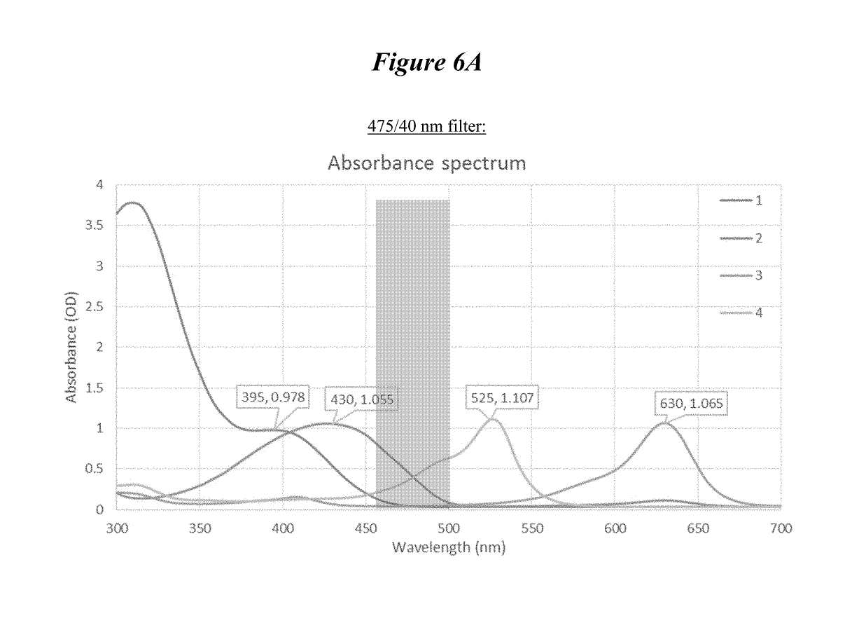

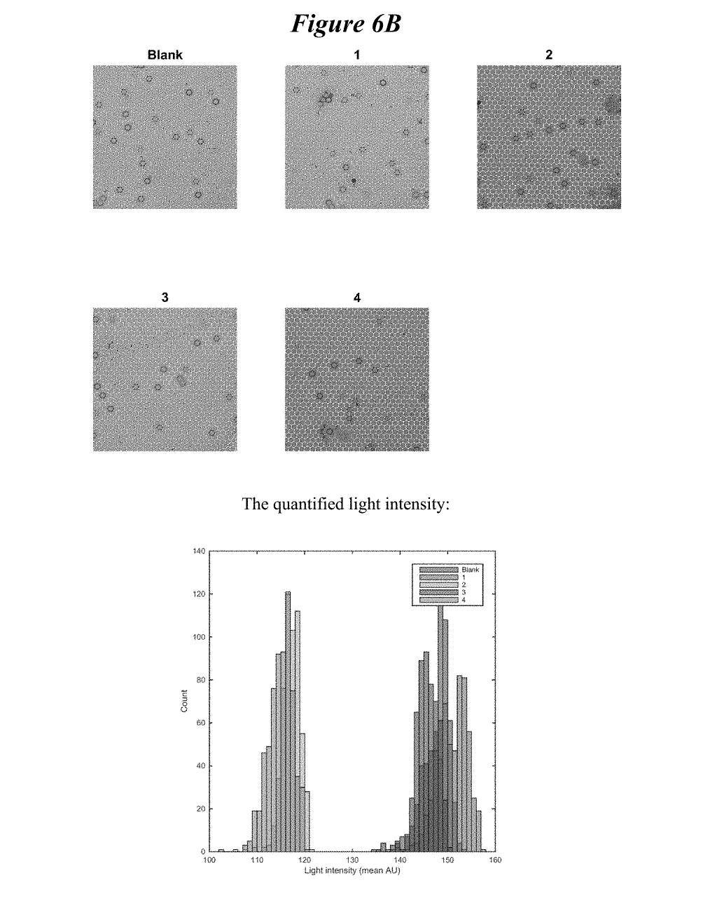

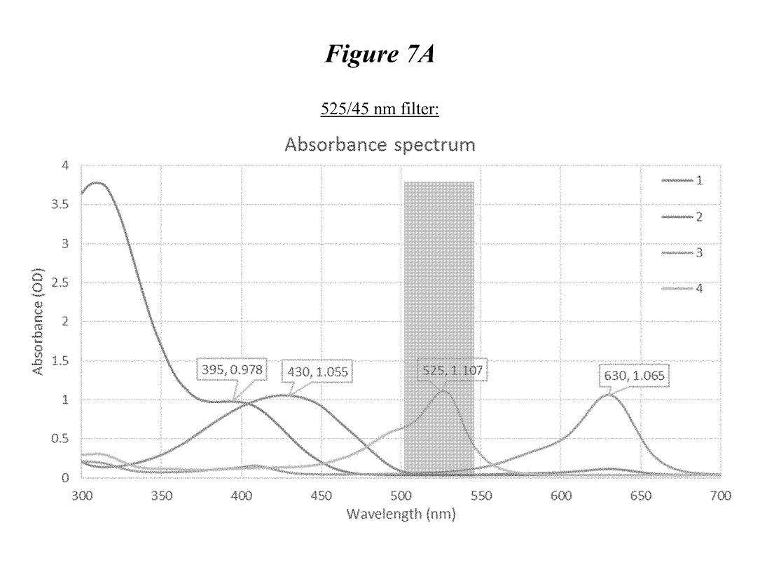

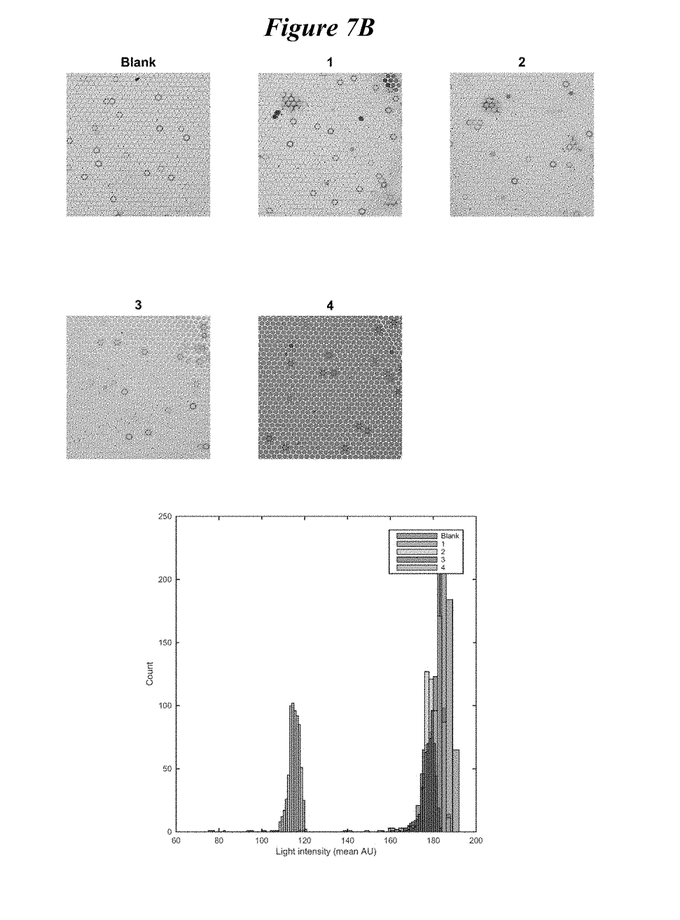

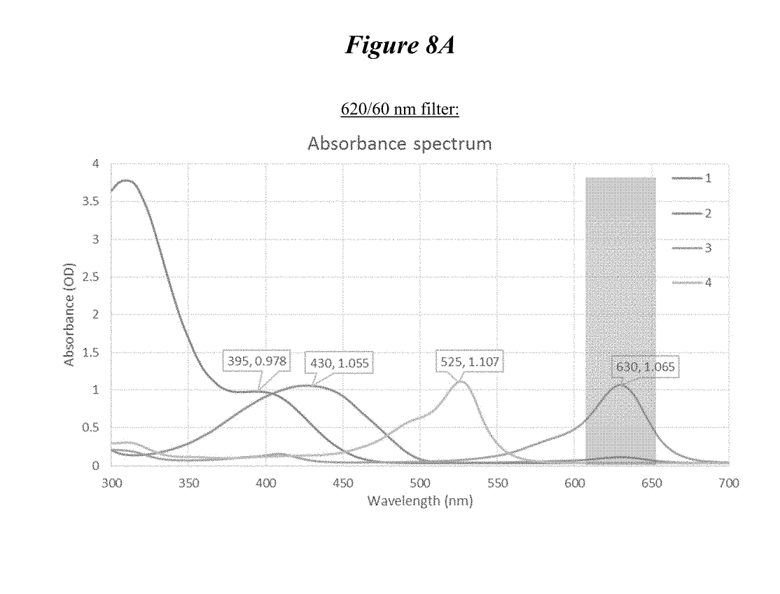

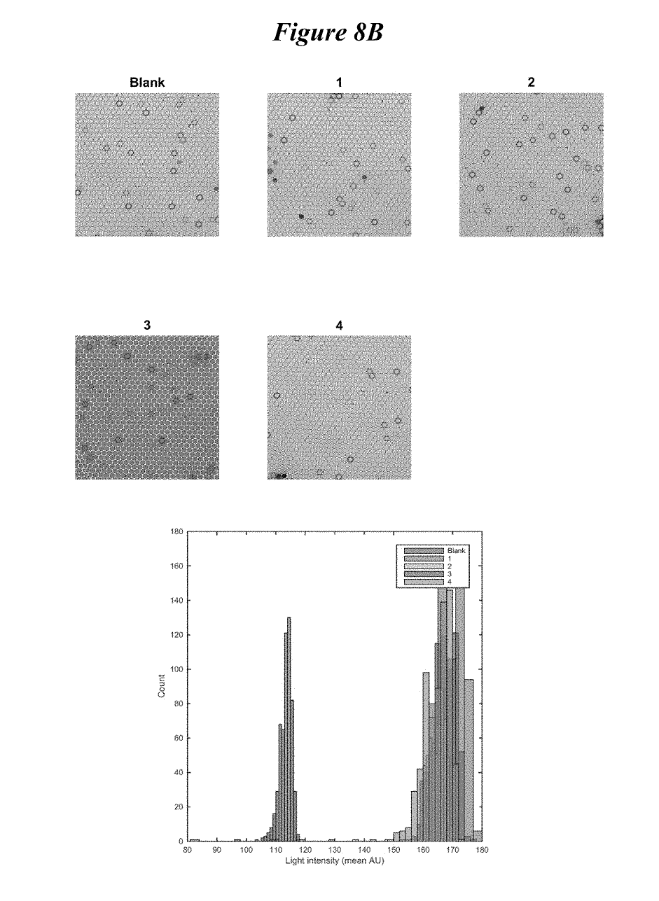

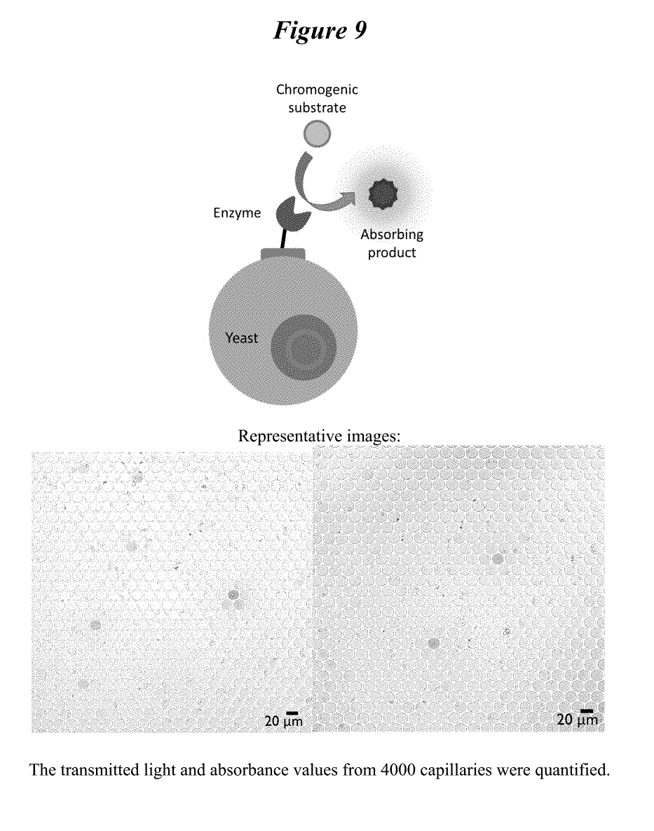

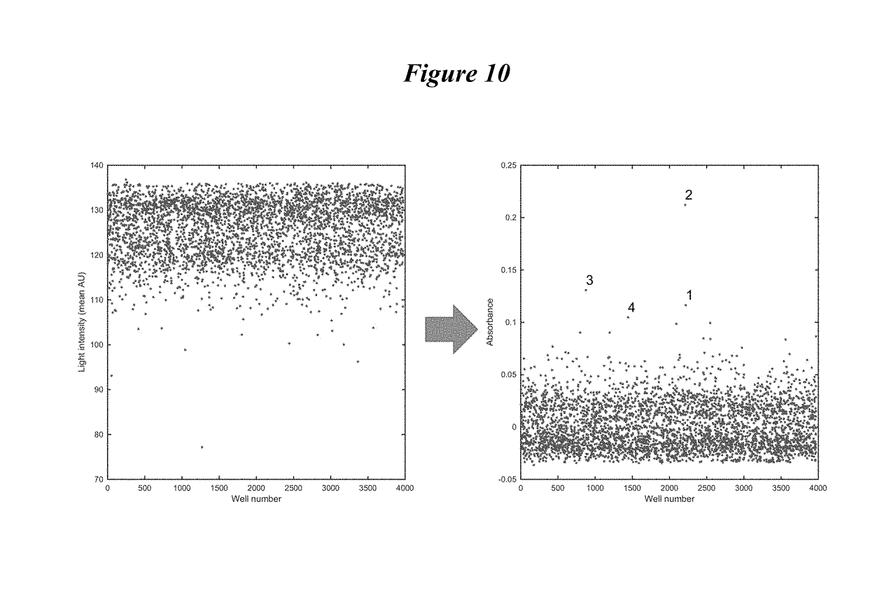

The 350/50 nm filter cube data is provided in FIG. 5A-5B. The 475/40 nm filter cube data is provide in FIG. 6A-5B. The 525/45 nm filter cube data is provided in FIG. 7A-7B. The 620/60 nm filter cube data is provide in FIG. 8A-8B.

Using the 350/50 nm filter cube Sample 1 and sample 2 show absorbance (lower light intensity), and minimal absorbance is shown in samples 3 and 4. See FIG. 5A.

Using the 475/40 nm filter cube, sample 2 and sample 4 show absorbance, and minimal absorbance is shown in sample 1 and 3. See FIG. 6A.

Using the 525/45 nm filter cube, sample 4 shows absorbance, and minimal absorbance is shown in samples 1, 2, and 3. See FIG. 7A.

Using the 620/60 nm filter cube, sample 3 shows absorbance, and minimal absorbance is shown in sample 1, 2, and 4. See FIG. 8A.

Demonstration of Absorbance with Diverse Enzyme Sample

To fully demonstrate the utility of method, a microcapillary array was loaded with various yeast displayed enzyme variants and a chromogenic substrate. These enzyme variants are displayed on the surface of yeast and will convert the chromogenic substrate to a substrate that absorbs at a given wavelength. Using this method, one would be able to select the variants that are more active.

The cells were mixed with a chromogenic substrate and diluted to a concentration which results in an average of 1 cells/capillary. After the 4 hours of enzymatic reaction, the microcapillary array was imaged using 350+/−50 nm filter.

The transmitted light and absorbance values from 4000 capillaries were quantified. 4 high absorbance variants were highlighted and further examined and shown below. Capillary 3 contained a bubble (a false positive) is not shown.

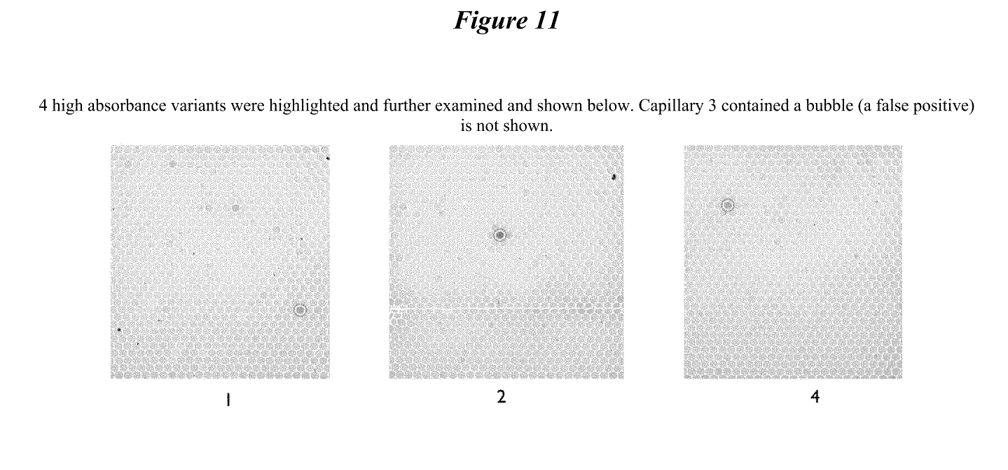

These 3 highlighted capillaries in theory contain cells that have higher activity or higher expression, which are desirable properties (see, FIG. 11). Overall, these experiments demonstrate a high-throughput way to discriminate a large number of enzyme variants via absorbance.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A high-throughput method for determining the absorbance for multiple samples in a microcavity array, the method comprising:
i) transmitting light of a definable wavelength through samples contained in said microcavity array, wherein one sample is loaded into each microcavity within the array;
ii) measuring the light transmitted through said samples with a detector, wherein the light transmitted is measured for each individual sample within the array in order to obtain a light transmittance intensity for each individual sample within the array;
iii) comparing the light transmittance intensity obtained for each individual sample in step ii) to the light transmittance intensity for a control sample; and
iv) calculating the absorbance of each individual sample in the array based on the comparison in step iii) in order to determine spectrometric differences between said samples.

2. The method of claim 1, wherein the light transmittance intensity is measured by the following formula:
$$T = \frac{{Intensity}_{sample}}{{Intensity}_{control}} .$$

3. The method of claim 1, wherein the light transmittance intensity is measured by the following formula:
$$T \% = \frac{{Intensity}_{sample}}{{Intensity}_{control}} * 100.$$

4. The method of claim 1, wherein the light transmittance intensity is measured by the following formula:
$$T = \frac{{Intensity}_{sample}}{{Intensity}_{average}} .$$

5. The method of claim 1, wherein the light transmittance intensity is measured by the following formula:
$$T \% = \frac{{Instensity}_{sample}}{{Intensity}_{average}} * 100.$$

6. The method of claim 2, wherein the absorbance is calculated by the following formula:
A=−log₁₀T.

7. The method of claim 3, wherein the absorbance is calculated by the following formula:
A=2−log₁₀T%.

8. The method of claim 1, wherein said method further comprises using said absorbance to determine one or more spectrometric characteristics.

9. The method of claim 8, wherein said spectrometric characteristics are selected from the group consisting of concentration, enzyme activity, enzyme-substrate interaction, receptor-ligand binding, affinity binding, stability, and cell growth.

10. The method of claim 1, wherein said enzyme activity results are based on a colorimetric assay.

11. The method of claim 1, wherein said concentration or cell growth results are based on a densitometric assay.

12. The method of claim 10, wherein said colorimetric assay is an enzyme based light absorbing assay.

13. The method of claim 11, wherein said densitometric assay is an assay wherein light is blocked by one or more materials in the sample.

14. The method of claim 13, wherein said material comprises one or more proteins, polypeptides, nucleic acid, small molecules, dyes, and/or cells.

15. The method of claim 1, wherein said method further comprises loading one sample into each microcavity prior to light transmission in step i).

16. The method of claim 1, wherein said microcavity is a microcapillary or a microwell.

17. The method of claim 1, wherein said light transmitted through said sample is detected by a microscope objective detector.

18. The method of claim 1, wherein said transmitted light is generated by a light source with a selectable wavelength.

19. The method of claim 18, wherein said transmitted light source is a high power plasma light source.

20. The method of claim 18, wherein said light source is a monochromatic light source.

21. The method of claim 20, wherein said light source is coupled to a monochromator.

22. The method of claim 18, wherein said light source is coupled to one or more filters.

23. The method of claim 18, wherein said light source is coupled to 1, 2, 3, 4, 5, or 6 filters.

24. The method of claim 1, wherein said sample comprises a biological material.

25. The method of claim 24, wherein said sample comprises proteins, polypeptides, nucleic acid, and/or cells.

26. The method of claim 25, wherein said proteins or polypeptides are selected from the group consisting of enzymes, ligands, and receptors.

27. The method of claim 1, wherein said measurement in step ii) occurs simultaneously for all the samples.

28. The method of claim 1, wherein said detector is a camera.

29. The method of claim 28, wherein said camera is a black and white camera.

30. The method of claim 28, wherein said camera is a color camera.

31. The method of claim 1, wherein said detector is a photodiode.

32. The method of claim 1, wherein when said detector is a photodiode, said method further comprises imaging the location of each microcavity before or after step ii).

33. The method of claim 1, wherein said measurements in step ii) are performed in real time.

34. The method of claim 1, wherein said measurements in step ii) are performed on the same samples as part of a time course.

35. The method of claim 1, wherein said microarray comprises at least 100,000 samples.

36. The method of claim 1, wherein said sample volume is less than 500 nL.

37. The method of claim 1, wherein said method further comprises detecting more than spectrometric characteristics.

38. The method of claim 37, wherein said method further comprises detecting transmittance and fluorescence.

39. A high-throughput microscope system for use in measuring the absorbance for multiple samples in a microcavity array, the microscope system comprising:
i) a light source unit comprising at least one light source capable of transmitting light of a definable wavelength through samples contained in said microcavity array, wherein one sample is loaded into each microcavity within the array;
ii) a detection unit comprising at least one detector capable of detecting the light transmitted through said samples, wherein the light transmitted is measured for each individual sample within the array in order to obtain a light transmittance intensity for each individual sample within the array;
iii) an optical train for directing the one or more illumination and/or excitation lights from the light source unit to the sample and for directing the transmitted light from the sample to the detection unit; and
iv) a control unit for controlling the light source unit and the detection unit; wherein, optionally the control unit is capable of:
a) comparing the light transmittance intensity obtained for each individual sample in step ii) to the light transmittance intensity for a control sample; and
b) calculating the absorbance of each individual sample in the array based on the comparison in step a) in order to determine differences between said samples.

40.-56. (canceled)
